(19) United States
(12) Patent Application Publication
(10) Pub. No.: US 2013/0123323 A1
(43) Pub. Date: May 16, 2013

(54) FUNGICIDAL PYRAZOLES

(71) Applicants: Moumita Kar, Secunderabad (IN); Kanu Maganbhai Patel, Wilmington, DE (US)

(72) Inventors: Moumita Kar, Secunderabad (IN); Kanu Maganbhai Patel, Wilmington, DE (US)

(73) Assignee: E I DU PONT DE NEMOURS AND COMPANY, Wilmington, DE (US)

(21) Appl. No.: 13/811,939

(22) PCT Filed: Aug. 19, 2011
(86) PCT No.: PCT/US2011/048420

§ 371 Date: Jan. 24, 2013

(30) Foreign Application Priority Data
Aug. 19, 2010 (IN) PCT/IN2010/000549

(51) Int. Cl. A01N 43/56; C07D 231/12

(52) U.S. Cl. CPC A01N 43/56; C07D 231/12
USPC 514/406; 548/377.1

(57) ABSTRACT

Disclosed are compounds of Formula 1, including all geometric and stereoisomers, N-oxides, and salts thereof,

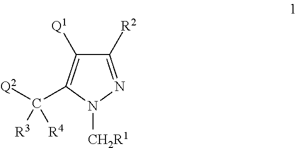

wherein
Q¹ is a phenyl ring, naphthalenyl ring system, a 5- to 6-membered fully unsaturated heterocyclic ring or an 8- to 10-membered heteroaromatic bicyclic ring system, each as described with optional substituents as defined in the disclosure;
Q² is a phenyl ring, a naphthalenyl ring system, a 5- to 6-membered saturated, partially unsaturated or fully unsaturated heterocyclic ring, or an 8- to 10-membered heteroaromatic bicyclic ring system, each as described with optional substituents as defined in the disclosure;
R³ is H, halogen or C₁-C₄ alkyl;
R⁴ is halogen;
and R¹ and R² are as defined in the disclosure.

Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain pyrazoles, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action. PCT Patent Publication WO 2009/137538 A2 discloses certain azole compounds as fungicides; however the fungicides of the present invention are not disclosed in this publication.

### SUMMARY OF THE INVENTION

This invention is directed to compounds of Formula 1 (including all stereoisomers), N-oxides, and salts thereof, agricultural compositions containing them and their use as fungicides:

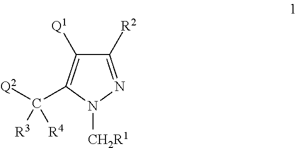

wherein
Q¹ is a phenyl ring or a naphthalenyl ring system, each ring or ring system optionally substituted with up to 5 substituents independently selected from R⁵; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2 S and up to 4 N atoms, wherein up to 3 carbon ring members are independently selected from C(═O) and C(═S), and the sulfur atom ring members are independently selected from S(═O)ᵤ(═NR⁶)ᵥ, each ring or ring system optionally substituted with up to 5 substituents independently selected from R⁵ on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
Q² is a phenyl ring or a naphthalenyl ring system, each ring or ring system optionally substituted with up to 5 substituents independently selected from R⁵; or a 5- to 6-membered saturated, partially unsaturated or fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2 S and up to 4 N atoms, wherein up to 3 carbon ring members are independently selected from C(═O) and C(═S), and the sulfur atom ring members are independently selected from S(═O)ᵤ(═NR⁶)ᵥ, each ring or ring system optionally substituted with up to 5 substituents independently selected from R⁵ on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C₁-C₁₂ alkyl, C₂-C₁₂ alkenyl, C₂-C₁₂ alkynyl, C₃-C₁₂ cycloalkyl or C₃-C₁₂ cycloalkenyl, each optionally substituted with up to 5 substituents independently selected from R⁵;
R¹ is H or CH₃;
R² is C₁-C₂ alkyl, halogen, cyano, cyanomethyl, halomethyl, hydroxymethyl, methoxy or methylthio; or cyclopropyl optionally substituted with up to 2 substituents independently selected from halogen and methyl;
R³ is H, halogen or C₁-C₄ alkyl;
R⁴ is halogen;
each R⁵ is independently selected from halogen, cyano, nitro, amino, methylamino, dimethylamino, formylamino, C₂-C₃ alkylcarbonylamino, C₁-C₄ alkyl, C₁-C₄ haloalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₂ alkylsulfonyloxy, C₁-C₂ haloalkylsulfonyloxy, C₃-C₄ cycloalkyl, C₃-C₇ cycloalkoxy, C₄-C₆ alkylcycloalkyl, C₄-C₆ cycloalkylalkyl, C₃-C₇ halocycloalkyl, C₂-C₄ alkenyl, C₂-C₄ alkynyl, hydroxy, formyl, C₂-C₃ alkylcarbonyl, C₂-C₃ alkylcarbonyloxy, —SF₅, —SCN, C(═S)NR⁷R⁸ or -U-V-T;
each R⁶ is independently H, cyano, C₁-C₃ alkyl or C₁-C₃ haloalkyl;
each R⁷ and R⁸ is independently H or CH₃;
each U is independently O, S(═O)_{w}, NR⁹ or a direct bond;
each V is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each T is independently cyano, NR^{10a}R^{10b}, OR¹¹ or S(═O)_{y}R¹²
each R⁹ is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{10a} and R^{10b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{10a} and R^{10b} attached to the same nitrogen atom are taken together with the nitrogen atom to form a 3- to 6-membered heterocyclic ring, the ring optionally substituted with up to 5 substituents independently selected from R¹³;
each R¹¹ and R¹² is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R¹³ is independently halogen, C₁-C₆ alkyl, C₁-C₆ haloalkyl or C₁-C₆ alkoxy;
each u and v are independently 0, 1 or 2 in each instance of S(═O)ᵤ(═NR⁶)ᵥ, provided that the sum of u and v is 0, 1 or 2;
each w is independently 0, 1 or 2; and
each y is independently 0, 1 or 2.

More particularly, this invention pertains to a compound selected from compounds of Formula 1 (including all stereoisomers) and N-oxides and salts thereof.

This invention also relates to a fungicidal composition comprising a compound of Formula 1, an N-oxide, or a salt thereof, and at least one additional component selected from the group consisting of surfactants, solid diluents and liquid diluents.

This invention also relates to a fungicidal composition comprising: (a) a compound of Formula 1, an N-oxide, or a salt thereof, and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

This invention also relates to a composition comprising a compound of Formula 1, an N-oxide, or a salt thereof, and at least one invertebrate pest control compound or agent.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of:”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

Generally when a molecular fragment (i.e. radical) is denoted by a series of atom symbols (e.g., C, H, N, O, S) the implicit point or points of attachment will be easily recognized by those skilled in the art. In some instances herein, particularly when alternative points of attachment are possible, the point or points of attachment may be explicitly indicated by a hyphen (“-”). For example, “—SCN” indicates that the point of attachment is the sulfur atom (i.e. thiocyanato, not isothiocyanato).

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl isomers. “Alkenyl” also includes polyenes such as 1,2-propadienyl. “Alkynyl” includes straight-chain or branched alkynes such as ethynyl, 1-propynyl, 2-propynyl and the different butynyl isomers. “Alkenylene” denotes a straight-chain or branched alkenediyl containing one olefinic bond. Examples of “alkenylene” include CH═CH, CH₂CH═CH, CH═C(CH₃). “Alkynylene” denotes a straight-chain or branched alkynediyl containing one triple bond. Examples of “alkynylene” include CH₂C≡C, C≡CCH₂ and the different butynylene, pentynylene and hexynylene isomers.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl isomers. Examples of “alkylaminoalkyl” include CH₃NHCH₂—, (CH₃)₂CHNHCH₂— and CH₃NHCH(CH₃)—. “Dialkylaminoalkyl” denotes two independent straight-chain or branched alkyl moieties bonded to a nitrogen atom of an amino(straight-chain or branched)alkyl moiety. Examples of “dialkylaminoalkyl” include (CH₃)₂NCH₂—, (CH₃)₂CH(CH₃)NCH₂— and (CH₃)₂NCH(CH₃)—. The term “alkylcarbonylamino” denotes alkyl bonded to a C(═O)NH moiety. Examples of “alkylcarbonylamino” include CH₃CH₂C(═O)NH and CH₃CH₂CH₂C(═O)NH.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkoxy” denotes cycloalkyl linked through an oxygen atom such as cyclopentyloxy and cyclohexyloxy. “Cycloalkenyl” includes carbocyclic rings that contain only one double bond such as cyclopentenyl and cyclohexenyl, as well as carbocyclic rings with more than one double bond such as 1,3- and 1,4-cyclohexadienyl, but are not aromatic. “Cycloalkylcarbonyl” denotes cycloalkyl bonded to a C(═O) group including, for example, cyclopropylcarbonyl and cyclopentylcarbonyl. The term “cycloalkoxycarbonyl” means cycloalkoxy bonded to a C(═O) group, for example, cyclopropyloxycarbonyl and cyclopentyloxycarbonyl. The term “cycloalkylene” denotes a cycloalkanediyl ring. Examples of “cycloalkylene” include cyclopropylene, cyclobutylene, cyclopentylene and cyclohexylene. The term“cycloalkenylene” denotes a cycloalkenediyl ring containing one olefinic bond. Examples of “cycloalkenylene” include cylopropenediyl and cyclpentenediyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “halocycloalkyl”, “haloalkoxy”, “haloalkylthio”, and the like, are defined analogously to the term “haloalkyl”. Examples of “haloalkoxy” include CH₂FO—, CHF₂O—, CF₃O—, CCl₃CH₂O—, HCF₂CH₂CH₂O— and CF₃CH₂O—. Examples of “fluoroalkoxy” include CH₂FO—, CHF₂O—, CF₃O—HCF₂CH₂CH₂O— and CF₃CH₂O—. Examples of “fluoromethoxy” include CH₂FO—, CHF₂O— and CF₃O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—.

The total number of carbon atoms in a substituent group is indicated by the “Cᵢ-Cⱼ” prefix where i and j are numbers from 1 to 12. For example, C₁-C₃ alkylsulfonyl designates methylsulfonyl through propylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

The term “unsubstituted” in connection with a group such as a ring or ring system means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. The number of optional substituents may be restricted by an expressed limitation. For example, the phrase “optionally substituted with up to 5 substituents independently selected from R⁵” means that 0, 1, 2, 3, 4 or 5 substituents can be present (if the number of potential connection points allows). When a range specified for the number of substituents (e.g., r being an integer from 0 to 4 or from 0 to 3 for 5- and 6-membered nitrogen-containing heterocycles in Exhibit A) exceeds the number of positions available for substituents on a ring (e.g., 2 positions available for (R^{a})ᵣ on U-27 in Exhibit A), the actual higher end of the range is recognized to be the number of available positions.

When a molecular structure is substituted with a substituent bearing a subscript that indicates the number of said substituents can exceed 1 (e.g., (R^{a})ᵣ in H-1 of Exhibit 1), said substituents (when they exceed 1) are independently selected from the group of defined substituents. When a group contains a substituent which can be hydrogen, for example R¹, R³, R⁶, R⁷, R⁸ or R⁹, then when this substituent is taken as hydrogen, it is recognized that this is equivalent to said group being unsubstituted. When a variable group is shown to be optionally attached to a position, for example (R^{a})ᵣ in H-23 of Exhibit 1, wherein r may be 0, then hydrogen may be at the position even if not recited in the variable group definition. When one or more positions on a group are said to be “not substituted” or “unsubstituted”, then hydrogen atoms are attached to take up any free valency.

The variables “u”, “v”, “w” and “y” in the Summary of the Invention and corresponding parts of the patent specification relate to subscripts appearing to the right of atoms or other molecular fragments within parentheses and denote the integral number of instances present of the atoms or other molecular fragments within the parentheses. “u” and “v” relate to “S(═O)ᵤ(═NR⁶)ᵥ”, “w” relates to “S(═O)_{w}”, and “y” relates to “S(═O)_{y}R¹². For example, “w” being 0, 1 or 2 means that “S(═O)_{W}” can be “S”, “S(═O)” or “S(═O)₂”. “S(O)” has the same meaning as “S(═O)”, and “S(O)₂” has the same meaning as “S(═O)₂”.

Unless otherwise indicated, a “ring” as a component of Formula 1 is carbocyclic or heterocyclic. The term “ring system” as a component of Formula 1 denotes two fused rings (e.g., a phenyl ring fused to a pyridinyl ring to form quinolinyl). The term “ring member” refers to an atom or other moiety (e.g., O, S(O), S(O)₂ or S(═O)ᵤ(═NR⁶)ᵥ) forming the backbone of a ring or ring system.

The terms “heterocyclic ring” or “heterocycle” denote a ring or ring system in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. The term “saturated heterocyclic ring” refers to a heterocyclic ring containing only single bonds between ring members. In regards to degree of saturation, “a partially unsaturated heterocyclic ring” is intermediate between a saturated heterocyclic ring and a fully unsaturated heterocyclic ring (which may be aromatic). Therefore, as referred to in the present disclosure and claims, the term “partially unsaturated heterocyclic ring” denotes a heterocyclic ring comprising at least one ring member bonded to an adjacent ring member through a double bond and which conceptually potentially accommodates a number of non-cumulated double bonds between adjacent ring members (i.e. in its fully unsaturated counterpart form) greater than the number of double bonds present (i.e. in its partially unsaturated form). When a fully unsaturated heterocyclic ring satisfies Hückel's rule, then said ring is also called a “heteroaromatic ring” or “aromatic heterocyclic ring”. The terms “heteroaromatic ring system” and “heteroaromatic bicyclic ring system” denote a ring system in which at least one atom forming the ring backbone is not carbon, e.g., nitrogen, oxygen or sulfur, and at least one ring is aromatic. Unless otherwise indicated, heterocyclic rings and ring systems can be attached through any available carbon or nitrogen by replacement of a hydrogen on said carbon or nitrogen.

“Aromatic” indicates that each of the ring atoms is essentially in the same plane and has a p-orbital perpendicular to the ring plane, and that (4n+2)π electrons, where n is a positive integer, are associated with the ring to comply with Hückel's rule. The term “aromatic heterocyclic ring system” denotes a heterocyclic ring system in which at least one ring of the ring system is aromatic.

In the context of the present invention when an instance of Q¹ or Q² comprises a phenyl or a 6-membered fully unsaturated heterocyclic ring, the ortho, meta and para positions of each ring is relative to the connection of the ring to the remainder of Formula 1.

As noted above, Q¹ and Q² can be (among others) phenyl optionally substituted with one or more substituents selected from a group of substituents as defined in the Summary of the Invention.

As noted above, Q¹ is, inter alia, a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2 S and up to 4 N atoms, wherein up to 3 carbon atom ring members are independently selected from C(═O) and C(═S), the sulfur atom ring members are independently selected from S(═O)ᵤ(═NR⁶)ᵥ, each ring or ring system optionally substituted with up to 5 substituents independently selected from any substituent defined in the Summary of the Invention for Q¹ (e.g., a Q¹ ring or ring system is optionally substituted with R⁵ on carbon ring members and cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members). Similarly, Q² is, inter alia, a 5- to 6-membered saturated, partially unsaturated or fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2 S and up to 4 N atoms, wherein up to 3 carbon atom ring members are independently selected from C(═O) and C(═S), the sulfur atom ring members are independently selected from S(═O)ᵤ(═NR⁶)ᵥ, each ring or ring system optionally substituted with up to 5 substituents independently selected from any substituent defined in the Summary of the Invention for Q². As the substituents on the ring or ring system of Q¹ or Q² are optional, 0 to 5 substituents may be present, limited only by the number of available points of attachment. In these definitions of heterocyclic ring and heteroaromatic ring system, the ring members selected from up to 2 O, up to 2 S and up to 4 N atoms are optional, provided at least one ring member is not carbon (e.g., N, O or S). The definition of S(═O)ᵤ(═NR⁶)ᵥ allows the up to 2 sulfur ring members, to be oxidized sulfur moieties (e.g., S(═O) or S(═O)₂) or unoxidized sulfur atoms (i.e. when u and v are both zero). The nitrogen atom ring members may be oxidized as N-oxides, because compounds relating to Formula 1 also include N-oxide derivatives. The up to 3 carbon atom ring members selected from C(═O) and C(═S) are in addition to the up to 4 heteroatoms selected from up to 2 O, up to 2 S and up to 4 N atoms.

The ring or ring system of Q¹ or Q² may be attached to the remainder of Formula 1 through any available carbon or nitrogen ring atom, unless otherwise described.

Examples of a 5- to 6-membered fully unsaturated heterocyclic ring include the rings H-1 through H-39 illustrated in Exhibit 1, and examples of an 8- to 10-membered heteroaromatic bicyclic ring system include the ring systems B-1 through B-39 illustrated in Exhibit 2. In Exhibits 1 and 2 the variable R^{a} is any substituent as defined in the Summary of the Invention for Q¹ or Q² (e.g., a Q¹ ring or ring system is optionally substituted with R⁵ on carbon ring members and cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members) and r is an integer from 0 to 5 for Q¹ and Q², limited by the number of available positions on each depicted ring or ring system.

### Exhibit 1

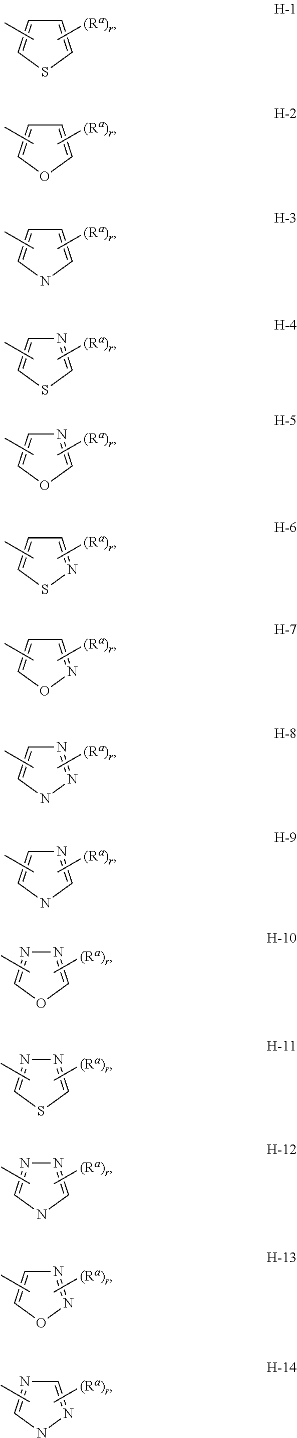

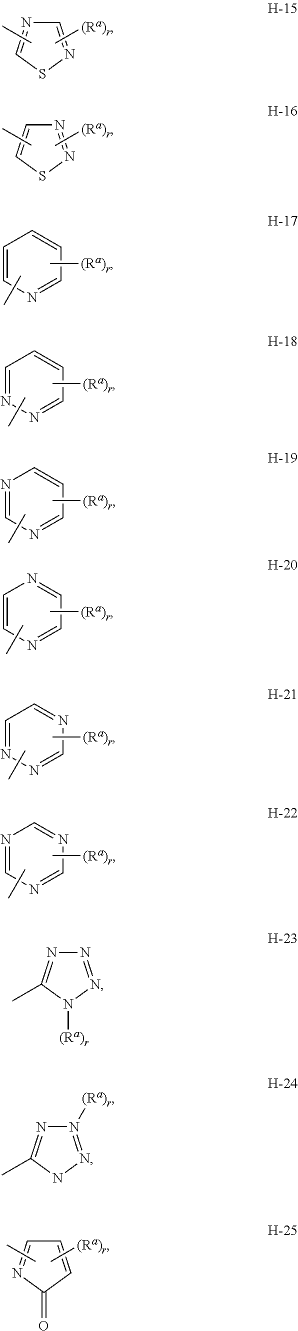

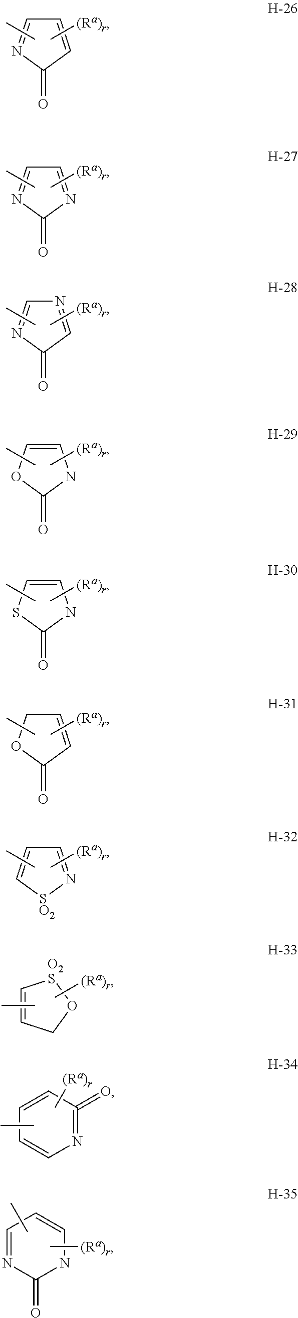

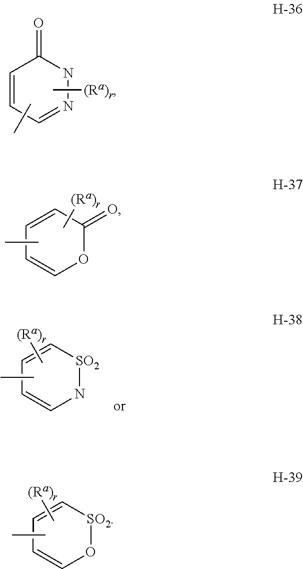

### Exhibit 2

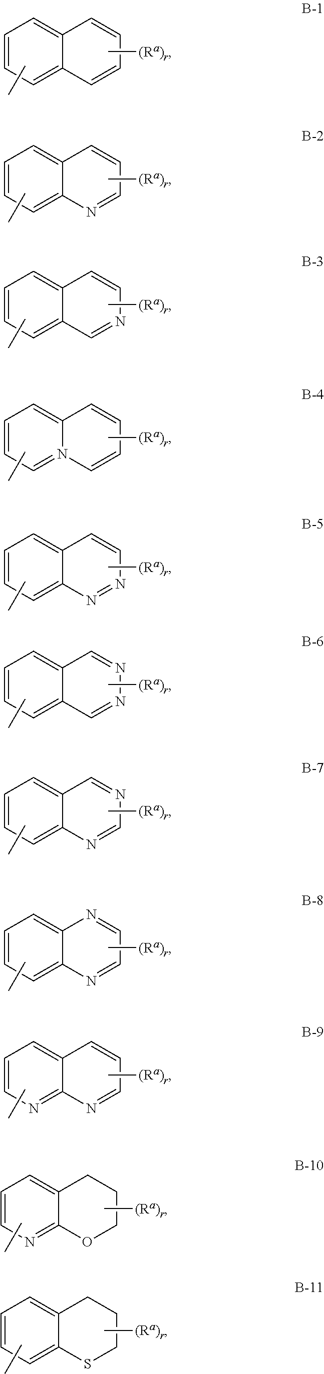

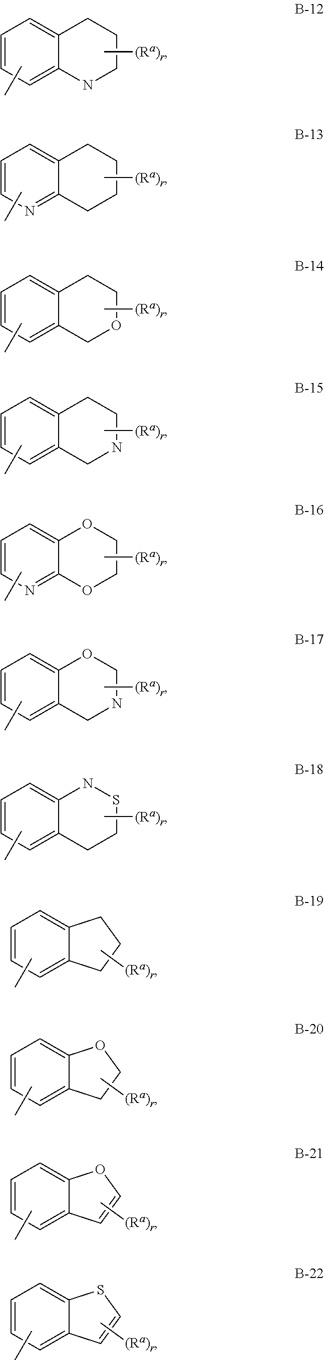

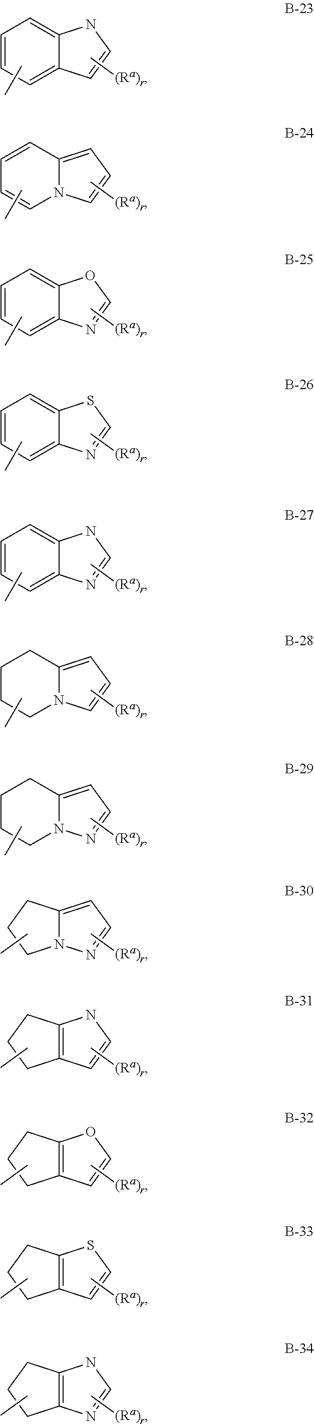

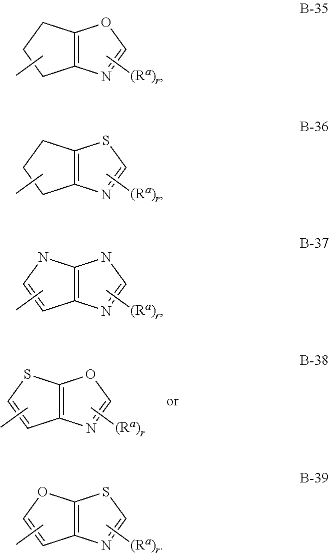

Examples of a saturated or partially unsaturated 5- to 6-membered fully unsaturated heterocyclic ring include the rings P-1 through P-40 illustrated in Exhibit 3. In Exhibit 3 the variable R^{a} is any substituent as defined in the Summary of the Invention for Q² (e.g., a Q² ring is optionally substituted with R⁵ on carbon ring members and cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members) and r is an integer from 0 to 5, limited by the number of available positions on each depicted ring or ring system.

### Exhibit 3

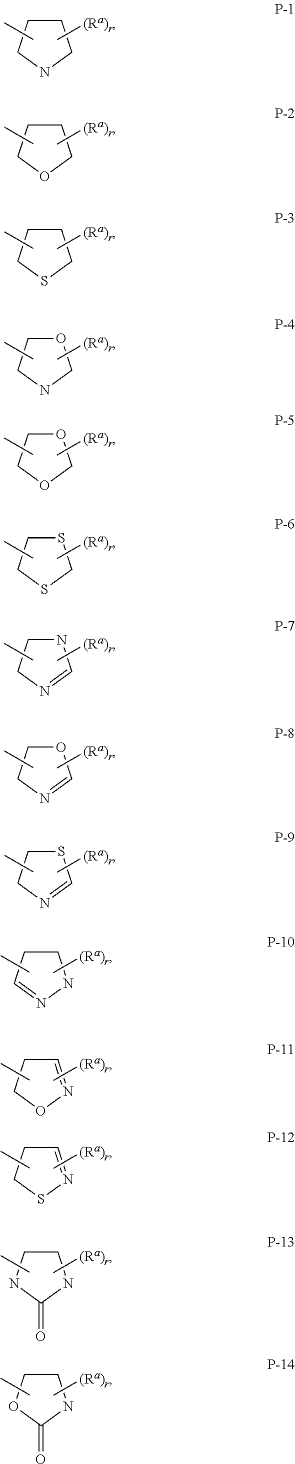

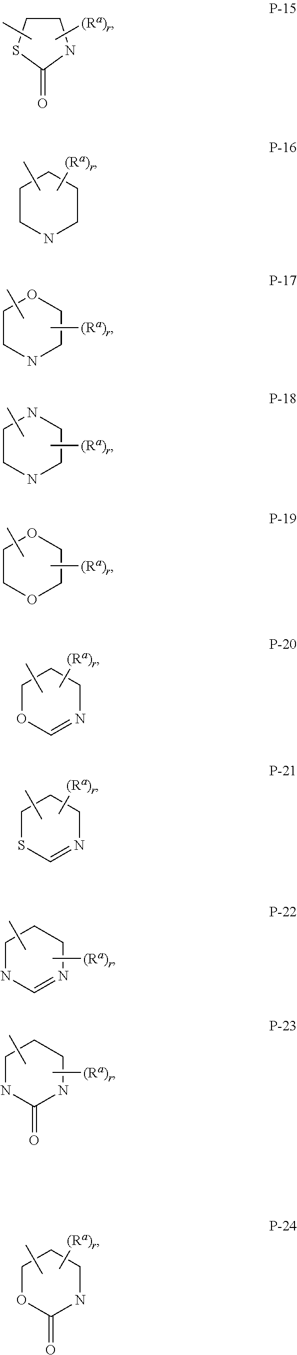

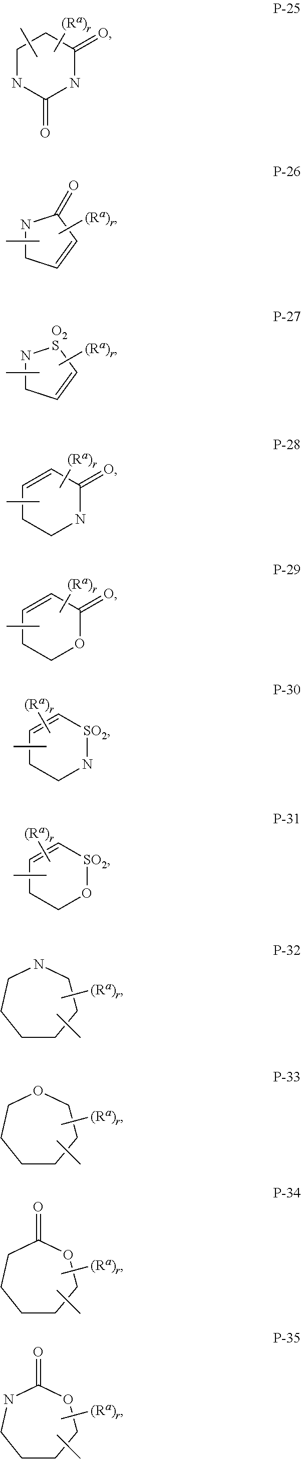

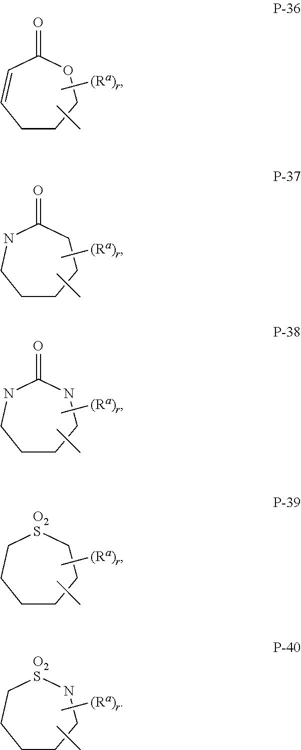

Examples of a 5- or 6-membered nitrogen-containing heterocycle optionally substituted with from one or more substituents of particular note for Q¹, Q² and R¹ include the rings U-1 through U-56 illustrated in Exhibit A wherein R^{a} is any substituent as defined in the Summary of the Invention for Q¹ or Q² (i.e. for Q¹ and Q²: R⁵ on carbon atom ring members, and the recited list of possible substituents on nitrogen atom ring members) and r is an integer ranging from 0 to 4 for Q¹ and Q², limited by the number of available positions on each U group. Note that some U groups can only be substituted with less than 4 R^{a} groups (e.g., U-4 through U-43 and U-47 through U-56). As U-24, U-25, U-31, U-32, U-33, U-34, U-35, U-36, U-37 and U-38 have only one available position, for these U groups, r is limited to the integers 0 or 1, and r being 0 means that the U group is unsubstituted and a hydrogen is present at the position indicated by (R^{a})ᵣ.

### Exhibit A

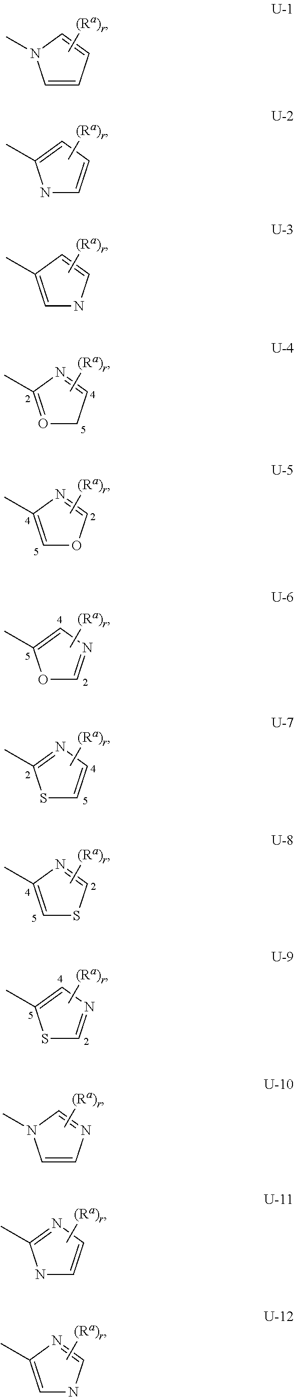

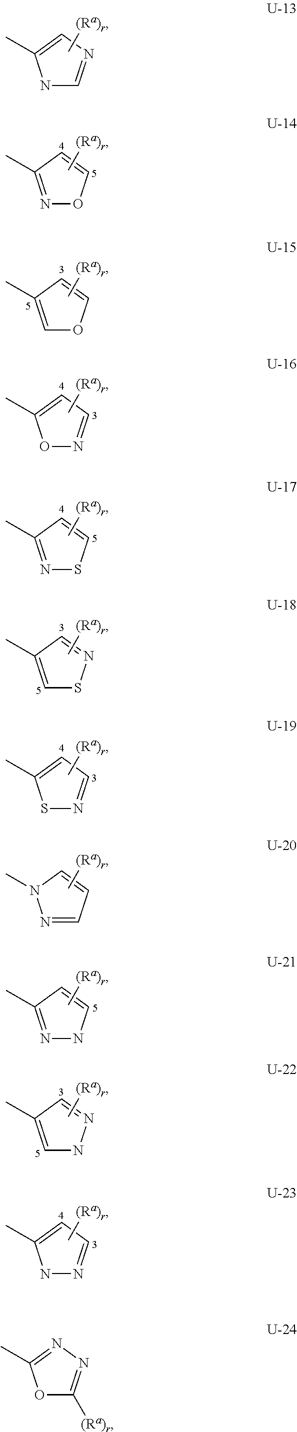

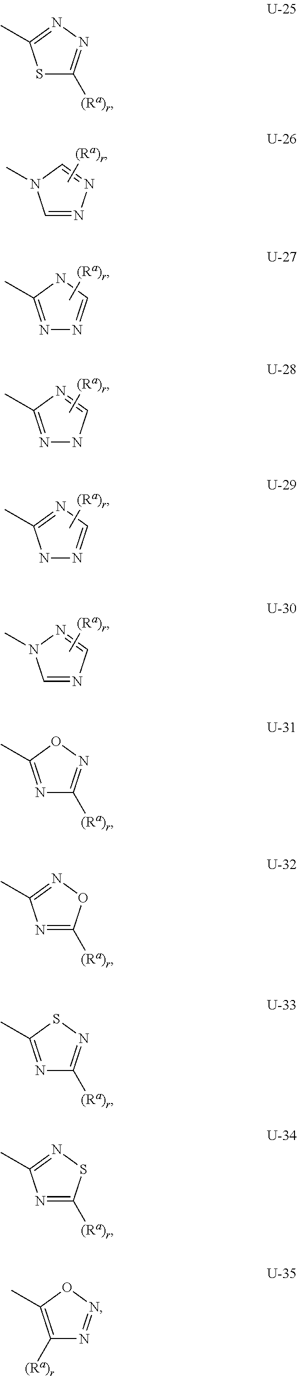

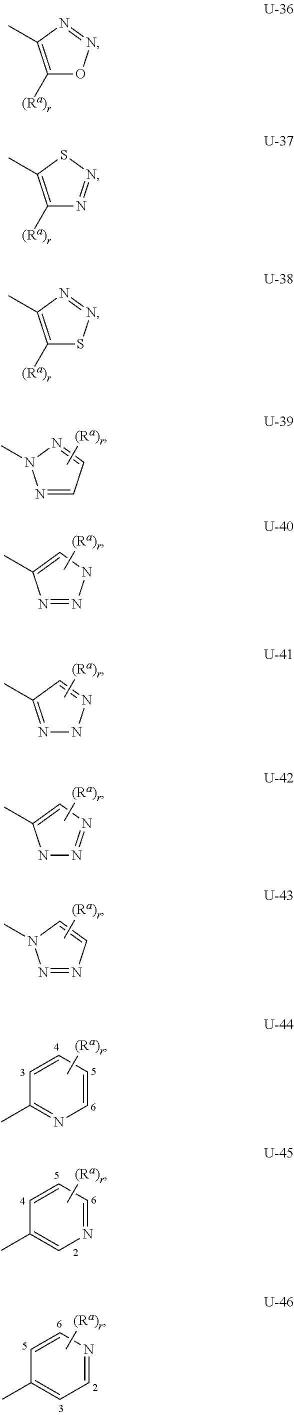

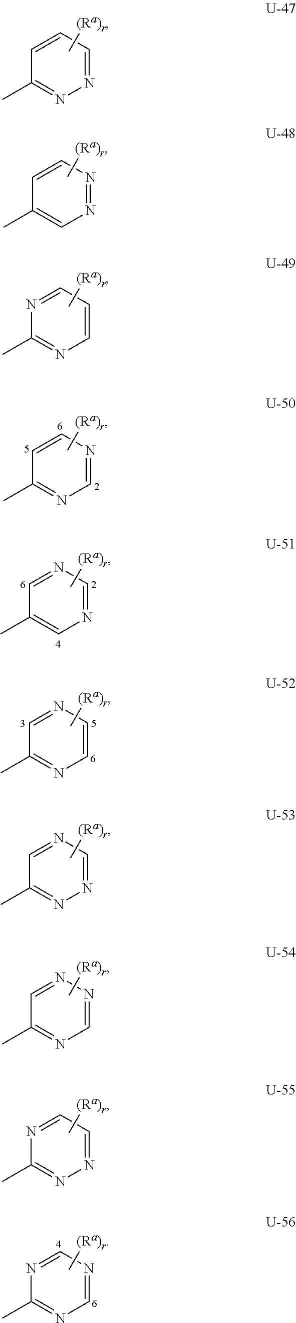

Although R^{a} groups are shown in the structures H-1 through H-39, B-1 through B-39, P-1 through P-40, and U-1 through U-57 in Exhibits 1 through 3 and Exhibit A, it is noted that they do not need to be present since they are optional substituents. The nitrogen atoms that require substitution to fill their valence are substituted with H or R^{a}. Note that when the attachment point between (R^{a})ᵣ and the H, B, P or U group in Exhibits 1 through 3 and Exhibit A is illustrated as floating, (R^{a})ᵣ can be attached to any available carbon atom or nitrogen atom of the H, B, P or U group. Note that when the attachment point on the H, B or P group in Exhibits 1 through 3 is illustrated as floating, the H, B or P group can be attached to the remainder of Formula 1 through any available carbon or nitrogen of the H, B or P group by replacement of a hydrogen atom.

Examples of where a pair of R^{10a} and R^{10b} attached to the same nitrogen atom are taken together with the nitrogen atom to form a 3- to 6-membered nonaromatic heterocyclic ring, the ring optionally substituted with up to 5 substituents independently selected from R¹³, include the rings G-1 through G-26 as illustrated in Exhibit 4.

### Exhibit 4

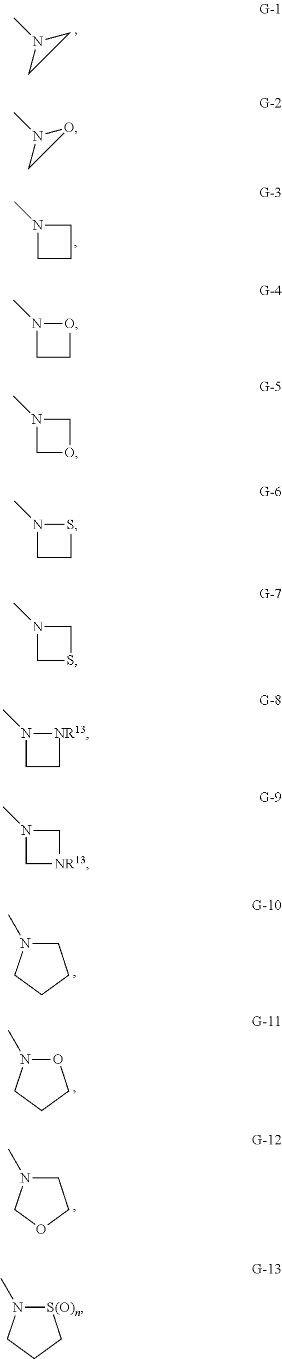

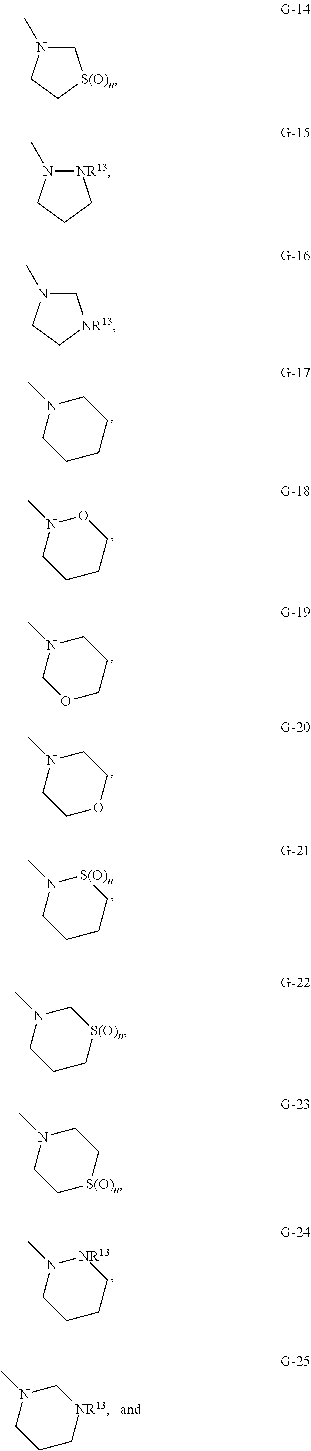

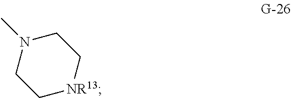

wherein n is 0, 1 or 2.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form. For example, two possible enantiomers of Formula 1 are depicted as Formula 1′ and Formula 1″ involving the chiral center identified with an asterisk (*) wherein R³ and R⁴ are not identical.

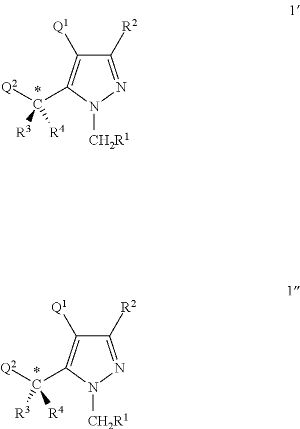

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

This invention comprises racemic mixtures, for example, equal amounts of the enantiomers of Formulae 1′ and 1″. In addition, this invention includes compounds that are enriched compared to the racemic mixture in an enantiomer of Formula 1. Also included are the essentially pure enantiomers of compounds of Formula 1, for example, Formula 1′ and Formula 1″.

When enantiomerically enriched, one enantiomer is present in greater amounts than the other, and the extent of enrichment can be defined by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers).

Of note are compositions of this invention having at least a 50%, or at least a 75%, or at least a 90%, or at least a 94% enantiomeric excess of an isomer. Of particular note are enantiomerically pure embodiments.

Compounds of Formula 1 can comprise additional chiral centers. For example, substituents such as R⁵ may themselves contain chiral centers.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

Compounds selected from Formula 1, geometric and stereoisomers, tautomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.

### Embodiment 1

A compound of Formula 1 wherein R³ is H, F, Cl, Br or CH₃.

### Embodiment 2

A compound of Embodiment 1 wherein R³ is H, F, Cl or CH₃.

### Embodiment 3

A compound of Embodiment 1 wherein R³ is H, F, Cl or Br.

### Embodiment 4

A compound of Embodiment 3 wherein R³ is H, F or Cl.

### Embodiment 5

A compound of Embodiment 4 wherein R³ is H or F.

### Embodiment 6

A compound of Embodiment 5 wherein R³ is H.

### Embodiment 7

A compound of Formula 1 or any one of Embodiments 1 through 6 wherein R⁴ is F, Cl or Br.

### Embodiment 8

A compound of Embodiment 7 wherein R⁴ is F or Cl.

### Embodiment 9

A compound of Embodiment 8 wherein R⁴ is F.

### Embodiment 10

A compound of Embodiment 8 wherein R⁴ is Cl.

### Embodiment 11

A compound of Formula 1 or any one of Embodiments 1 through 10 wherein R¹ is H.

### Embodiment 12

A compound of Formula 1 or any one of Embodiments 1 through 11 wherein R² is C₁-C₂ alkyl, halogen, cyano, cyanomethyl, monohalomethyl, hydroxymethyl, methoxy or methylthio; or cyclopropyl optionally substituted with up to 2 substituents independently selected from halogen and methyl.

### Embodiment 13

A compound of Embodiment 12 wherein R² is C₁-C₂ alkyl, Cl, Br or I.

### Embodiment 14

A compound of Embodiment 13 wherein R² is C₁-C₂ alkyl, Cl or Br.

### Embodiment 15

A compound of Embodiment 14 wherein R² is CH₃, Cl or Br.

### Embodiment 16

A compound of Embodiment 15 wherein R² is CH₃ or Cl.

### Embodiment 17

A compound of Embodiment 16 wherein R² is CH₃.

### Embodiment 18

A compound of Embodiment 15 wherein R² is Cl or Br.

### Embodiment 19

A compound of Embodiment 18 wherein R² is Cl.

### Embodiment 20

A compound of Formula 1 or any one of Embodiments 1 through 19 wherein Q¹ is phenyl, thienyl, pyridinyl, pyridazinyl, pyrazinyl, pyrimidinyl, naphthalenyl, quinolinyl, isoquinolinyl or quinoxalinyl, each optionally substituted with up to 5 substituents independently selected from R⁵.

### Embodiment 21

A compound of Embodiment 20 wherein Q¹ is phenyl, thienyl, pyridinyl, pyridazinyl, pyrazinyl or pyrimidinyl, each optionally substituted with up to 5 substituents independently selected from R⁵.

### Embodiment 22

A compound of Embodiment 21 wherein Q¹ is phenyl, pyridinyl, pyrimidinyl, pyrazinyl or pyridazinyl, each substituted with from 1 to 4 substituents independently selected from R⁵.

### Embodiment 23

A compound of Embodiment 22 wherein Q¹ is phenyl, pyridinyl, pyrimidinyl, pyrazinyl or pyridazinyl, each substituted with 1, 2 or 3 substituents independently selected from R⁵.

### Embodiment 24

A compound of any one of Embodiments 21 through 23 wherein the substituents are located at the ortho and/or para positions (relative to the connection of the Q¹ ring to the remainder of Formula 1) of the phenyl, pyridinyl, pyrimidinyl, pyrazinyl or pyridazinyl of Q¹.

### Embodiment 25

A compound of Embodiment 23 or 24 wherein Q¹ is phenyl or pyridinyl, each substituted with 1, 2 or 3 substituents independently selected from R⁵.

### Embodiment 26

A compound of Embodiment 25 wherein Q¹ is phenyl or pyridinyl, each substituted with 2 or 3 substituents independently selected from R⁵.

### Embodiment 27

A compound of Embodiment 26 wherein Q¹ is phenyl substituted at the 2-, 4- and 6-positions with substituents independently selected from R⁵; or phenyl substituted at the 2- and 4-positions with substituents independently selected from R⁵; or phenyl substituted at the 2- and 6-positions with substituents independently selected from R⁵.

### Embodiment 28

A compound of Embodiment 27 wherein Q¹ is phenyl substituted at the 2-, 4- and 6-positions with substituents independently selected from R⁵.

### Embodiment 29

A compound of Embodiment 27 wherein Q¹ is phenyl substituted at the 2- and 4-positions with substituents independently selected from R⁵.

### Embodiment 30

A compound of Embodiment 27 wherein Q¹ is phenyl substituted at the 2- and 6-positions with substituents independently selected from R⁵.

### Embodiment 31

A compound of Embodiment 25 wherein Q¹ is pyridinyl substituted with 1, 2 or 3 substituents independently selected from R⁵.

### Embodiment 32

A compound of Embodiment 31 wherein Q¹ is pyridinyl substituted with 1 or 2 substituents independently selected from R⁵.

### Embodiment 33

A compound of Embodiment 32 wherein Q¹ is pyridinyl substituted with 1 substituent independently selected from R⁵.

### Embodiment 34

A compound of Formula 1 or any one of Embodiments 1 through 33 wherein when Q¹ is a six-membered ring substituted with only one R⁵ substituent, then said R⁵ substituent is attached at an ortho position (relative to the connection of the Q¹ ring to the remainder of Formula 1).

### Embodiment 35

A compound of Formula 1 or any one of Embodiments 1 through 34 wherein Q² is phenyl, thienyl, pyridinyl, pyridazinyl, pyrazinyl, pyrimidinyl, naphthalenyl, quinolinyl, isoquinolinyl or quinoxalinyl, each optionally substituted with up to 5 substituents independently selected from R⁵.

### Embodiment 36

A compound of Embodiment 35 wherein Q² is phenyl, thienyl, pyridinyl, pyridazinyl, pyrazinyl or pyrimidinyl, each optionally substituted with up to 5 substituents independently selected from R⁵.

### Embodiment 37

A compound of Embodiment 36 wherein Q² is phenyl, pyridinyl, pyrimidinyl, pyrazinyl or pyridazinyl, each substituted with from 1 to 4 substituents independently selected from R⁵.

### Embodiment 38

A compound of Embodiment 37 wherein Q² is phenyl, pyridinyl, pyrimidinyl, pyrazinyl or pyridazinyl, each substituted with 1, 2 or 3 substituents independently selected from R⁵.

### Embodiment 39

A compound of any one of Embodiments 36 through 38 wherein the substituents are located at the ortho and/or para positions (relative to the connection of the Q² ring to the remainder of Formula 1) of the phenyl, pyridinyl, pyrimidinyl, pyrazinyl or pyridazinyl of Q².

### Embodiment 40

A compound of any one of Embodiments 38 or 39 wherein Q² is phenyl or pyridinyl, each substituted with 1, 2 or 3 substituents independently selected from R⁵.

### Embodiment 41

A compound of Embodiment 40 wherein Q² is phenyl substituted with 1, 2 or 3 substituents independently selected from R⁵.

### Embodiment 42

A compound of Embodiment 41 wherein Q² is phenyl substituted at the 2-, 4- and 6-positions with substituents independently selected from R⁵; or phenyl substituted at the 2- and 4-positions with substituents independently selected from R⁵; or phenyl substituted at the 2- and 6-positions with substituents independently selected from R⁵.

### Embodiment 43

A compound of Embodiment 42 wherein Q² is phenyl substituted at the 2-, 4- and 6-positions with substituents independently selected from R⁵.

### Embodiment 44

A compound of Embodiment 42 wherein Q² is phenyl substituted at the 2- and 4-positions with substituents independently selected from R⁵.

### Embodiment 45

A compound of Embodiment 42 wherein Q² is phenyl substituted at the 2- and 6-positions with substituents independently selected from R⁵.

### Embodiment 46

A compound of Embodiment 40 wherein Q² is pyridinyl substituted with 1, 2 or 3 substituents independently selected from R⁵.

### Embodiment 47

A compound of Embodiment 46 wherein Q² is pyridinyl substituted with 1 or 2 substituents independently selected from R⁵.

### Embodiment 48

A compound of Embodiment 47 wherein Q² is pyridinyl substituted with 1 substituent selected from R⁵.

### Embodiment 49

A compound of Formula 1 or any one of Embodiments 1 through 48 wherein when Q² is a six-membered ring (e.g., phenyl, pyridinyl, pyrimidinyl, pyrazinyl or pyridazinyl) substituted with only one R⁵ substituent, then said R⁵ substituent is attached at an ortho position (relative to the connection of the Q² ring to the remainder of Formula 1).

### Embodiment 50

A compound of Formula 1 or any one of Embodiments 1 through 49 wherein at least one of Q¹ and Q² is phenyl optionally substituted with R⁵ (e.g., optionally substituted with up to 5 substituents independently selected from R⁵).

### Embodiment 51

A compound of Embodiment 50 wherein at least one of Q¹ and Q² is phenyl substituted with 2, 3 or 4 substituents independently selected from R⁵.

### Embodiment 52

A compound of Embodiment 51 wherein at least one of Q¹ and Q² is phenyl substituted with 2 or 3 substituents independently selected from R⁵.

### Embodiment 53

A compound of Embodiment 52 wherein each of Q¹ and Q² is phenyl substituted with 2 or 3 substituents independently selected from R⁵.

### Embodiment 54

A compound of Formula 1 or any one of Embodiments 1 through 53 wherein each R⁵ is independently selected from halogen, cyano, nitro, amino, methylamino, dimethylamino, C₁-C₄ alkyl, C₁-C₄ haloalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₃-C₄ cycloalkyl, C(═S)NH₂ and -U-V-T.

### Embodiment 55

A compound of Embodiment 54 wherein each R⁵ is independently selected from halogen, cyano, nitro, C₁-C₄ alkyl, C₁-C₄ haloalkyl, C₁-C₃ alkoxy, C₁-C₃ haloalkoxy and -U-V-T.

### Embodiment 56

A compound of Embodiment 55 wherein each R⁵ is independently selected from halogen, cyano, C₁-C₃ alkyl, C₁-C₃ haloalkyl, C₁-C₃ alkoxy, C₁-C₃ haloalkoxy and -U-V-T.

### Embodiment 57

A compound of Embodiment 56 wherein each R⁵ is independently selected from F, Cl, Br, cyano, C₁-C₂ alkyl, C₁-C₂ haloalkyl, C₁-C₂ alkoxy, C₁-C₂ haloalkoxy and -U-V-T.

### Embodiment 58

A compound of Embodiment 57 wherein each R⁵ is independently selected from F, Cl, Br, cyano, methyl, C₁-C₂ alkoxy, fluoromethoxy and -U-V-T.

### Embodiment 59

A compound of Formula 1 or any one of Embodiments 1 through 58 wherein at least one R⁵ substituent on the ring or ring system of Q¹ or Q² is -U-V-T.

### Embodiment 60

A compound of Formula 1 or any one of Embodiments 1 through 58 wherein each R⁵ is other than -U-V-T.

### Embodiment 61

A compound of Embodiment 58 wherein each R⁵ is independently selected from F, Cl, Br, cyano and methoxy.

### Embodiment 62

A compound of Embodiment 61 wherein each R⁵ is independently selected from F, Cl, Br and cyano.

### Embodiment 63

A compound of Embodiment 62 wherein each R⁵ is independently selected from F, Cl and cyano.

### Embodiment 64

A compound of Formula 1 or any one of Embodiments 1 through 59 wherein each U is independently O or NR⁹.

### Embodiment 65

A compound of Embodiment 64 wherein each U is independently O or NH.

### Embodiment 66

A compound of Formula 1 or any one of Embodiments 1 through 59 or 64 through 65 wherein each V is C₂-C₄ alkylene.

### Embodiment 67

A compound of Formula 1 or any one of Embodiments 1 through 59 or 64 through 66 wherein each T is independently NR^{10a}R^{10b} or OR¹¹.

### Embodiment 68

A compound of Formula 1 or any one of Embodiments 1 through 59 or 64 through 67 wherein each R^{10aa} and R^{10b} is independently H, C₁-C₆ alkyl or C₁-C₆ haloalkyl.

### Embodiment 69

A compound of Formula 1 or any one of Embodiments 1 through 59 or 64 through 68 wherein each R¹¹ is independently H, C₁-C₆ alkyl or C₁-C₆ haloalkyl.

### Embodiment 70

A compound of Formula 1 or any one of Embodiments 1 through 69 wherein when Q¹ is a six-membered ring (e.g., phenyl, pyridinyl, pyrimidinyl, pyrazinyl or pyridazinyl) and an R⁵ substituent is located at an ortho or meta position (relative to the connection of the Q¹ ring to the remainder of Formula 1), then said R⁵ substituent is selected from F, Cl, Br, cyano, methyl, C₁-C₂ alkoxy and fluoromethoxy.

### Embodiment 71

A compound of Formula 1 or any one of Embodiments 1 through 70 wherein when Q¹ is a six-membered ring (e.g., phenyl, pyridinyl, pyrimidinyl, pyrazinyl or pyridazinyl) and an R⁵ substituent is located at a meta position (relative to the connection of the Q¹ ring to the remainder of Formula 1), then said R⁵ substituent is selected from F, Cl, Br and cyano.

### Embodiment 72

A compound of Formula 1 or any one of Embodiments 1 through 71 wherein when Q¹ is a six-membered ring (e.g., phenyl, pyridinyl, pyrimidinyl, pyrazinyl or pyridazinyl) and an R⁵ substituent is located at a meta position (relative to the connection of the Q¹ ring to the remainder of Formula 1), then said R⁵ substituent is F.

### Embodiment 73

A compound of Formula 1 or any one of Embodiments 1 through 72 wherein when Q² is a six-membered ring (e.g., phenyl, pyridinyl, pyrimidinyl, pyrazinyl or pyridazinyl) and an R⁵ substituent is located at an ortho or meta position (relative to the connection of the Q² ring to the remainder of Formula 1), then said R⁵ substituent is selected from F, Cl, Br, cyano, methyl, C₁-C₂ alkoxy and fluoromethoxy.

### Embodiment 74

A compound of Formula 1 or any one of Embodiments 1 through 73 wherein when Q² is a six-membered ring (e.g., phenyl, pyridinyl, pyrimidinyl, pyrazinyl or pyridazinyl) and an R⁵ substituent is located at a meta position (relative to the connection of the Q² ring to the remainder of Formula 1), then said R⁵ substituent is selected from F, Cl, Br and cyano.

### Embodiment 75

A compound of Formula 1 or any one of Embodiments 1 through 74 wherein when Q² is a six-membered ring and an R⁵ substituent is located at a meta position (relative to the connection of the Q² ring to the remainder of Formula 1), then said R⁵ substituent is F.

Embodiments of this invention, including Embodiments 1-75 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-75 above as well as any other embodiments described herein, and any combination thereof, pertain to the compositions and methods of the present invention.

Combinations of Embodiments 1-75 are illustrated by:

### Embodiment A

A compound of Formula 1 wherein
Q¹ is phenyl, pyridinyl, pyrimidinyl, pyrazinyl or pyridazinyl, each substituted with from 1 to 4 substituents independently selected from R⁵; provided that when an R⁵ substituent is located at a meta position, then said R⁵ substituent is selected from F, Cl, Br and cyano;
Q² is phenyl, pyridinyl, pyrimidinyl, pyrazinyl or pyridazinyl, each substituted with 1, 2 or 3 substituents independently selected from R⁵, provided that when an R⁵ substituent is located at a meta position, then said R⁵ substituent is selected from F, Cl, Br and cyano;
R² is C₁-C₂ alkyl, Cl or Br; and
each R⁵ is independently selected from halogen, cyano, nitro, amino, methylamino, dimethylamino, C₁-C₄ alkyl, C₁-C₄ haloalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₃-C₄ cycloalkyl, C(═S)NH₂ and -U-V-T.

### Embodiment B

A compound of Embodiment A wherein
Q¹ is phenyl or pyridinyl, each substituted with 1, 2 or 3 substituents independently selected from R⁵;
Q² is phenyl or pyridinyl, each substituted with 1, 2 or 3 substituents independently selected from R⁵;
R² is CH₃, Cl or Br;
R³ is H, F, Cl, Br or CH₃;
R⁴ is F, Cl or Br;
each R⁵ is independently selected from halogen, cyano, nitro, C₁-C₄ alkyl, C₁-C₄ haloalkyl, C₁-C₃ alkoxy, C₁-C₃ haloalkoxy and -U-V-T;
each U is independently O or NH;
each V is C₂-C₄ alkylene;
each T is independently NR^{10a}R^{10b} or OR¹¹;
each R^{10a} and R^{10b} is independently H, C₁-C₆ alkyl or C₁-C₆ haloalkyl; and
each R¹¹ is independently H, C₁-C₆ alkyl or C₁-C₆ haloalkyl.

### Embodiment C

A compound of Embodiment B wherein
at least one of Q¹ and Q² is phenyl substituted with 2 or 3 substituents independently selected from R⁵;
R¹ is H;
R² is CH₃;
R³ is H, F, Cl or Br; and
each R⁵ is independently selected from halogen, cyano, C₁-C₃ alkyl, C₁-C₃ haloalkyl, C₁-C₃ alkoxy and C₁-C₃ haloalkoxy.

### Embodiment D

A compound of Embodiment C wherein
Q¹ is phenyl substituted at the 2-, 4- and 6-positions with substituents independently selected from R⁵; or phenyl substituted at the 2- and 4-positions with substituents independently selected from R⁵; or phenyl substituted at the 2- and 6-positions with substituents independently selected from R⁵;
Q² is phenyl substituted at the 2-, 4- and 6-positions with substituents independently selected from R⁵; or phenyl substituted at the 2- and 4-positions with substituents independently selected from R⁵; or phenyl substituted at the 2- and 6-positions with substituents independently selected from R⁵;
R³ is H, F or Cl;
R⁴ is F or Cl; and
each R⁵ is independently selected from F, Cl, Br, cyano, C₁-C₂ alkyl, C₁-C₂ haloalkyl, C₁-C₂ alkoxy and C₁-C₂ haloalkoxy.

### Embodiment E

A compound of Embodiment D wherein
R³ is H; and
each R⁵ is independently selected from F, Cl, Br, cyano, methyl, C₁-C₂ alkoxy and fluoromethoxy.

### Embodiment F

A compound of Embodiment E wherein
each R⁵ is independently selected from F, Cl, Br, cyano and methoxy.

Specific embodiments include compounds of Formula 1 selected from the group consisting of:
4-(2-chloro-4-fluorophenyl)-5-[(2,4-difluorophenyl)fluoromethyl]-1,3-dimethyl-1H-pyrazole,
5-[chloro(2,4-difluorophenyl)methyl]-4-(2-chloro-4-fluorophenyl)-1,3-dimethyl-1H-pyrazole,
5-[bromo(2,4-difluorophenyl)methyl]-4-(2-chloro-4-fluorophenyl)-1,3-dimethyl-1H-pyrazole,
4-(2-chloro-4-fluorophenyl)-5-[(2,4-difluorophenyl)difluoromethyl]-1,3-dimethyl-1H-pyrazole,
4-(2-chloro-4-fluorophenyl)-5-[dichloro (2,4-difluorophenyl)methyl]-1,3-dimethyl-1H-pyrazole,
4-(2-chloro-4-fluorophenyl)-5-[dibromo (2,4-difluorophenyl)methyl]-1,3-dimethyl-1H-pyrazole,
5-[chloro(2,4-difluorophenyl)fluoromethyl]-4-(2-chloro-4-fluorophenyl)-1,3-dimethyl-1H-pyrazole,
5-[1-chloro-1-(2,4-difluorophenyl)ethyl]-4-(2-chloro-4-fluorophenyl)-1,3-dimethyl-1H-pyrazole,
4-(2-chloro-4-fluorophenyl)-5-[1-(2,4-difluorophenyl)-1-fluoroethyl]-1,3-dimethyl-1H-pyrazole, and
5-[1-bromo-1-(2,4-difluorophenyl)ethyl]-4-(2-chloro-4-fluorophenyl)-1,3-dimethyl-1H-pyrazole.

This invention provides a fungicidal composition comprising a compound of Formula 1 (including all geometric and stereoisomers, N-oxides, and salts thereof), and at least one other fungicide. Of note as embodiments of such compositions are compositions comprising a compound corresponding to any of the compound embodiments described above.

This invention provides a fungicidal composition comprising a fungicidally effective amount of a compound of Formula 1 (including all geometric and stereoisomers, N-oxides, and salts thereof), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers,
N-oxides, and salts thereof). Of note as embodiments of such methods are methods comprising applying a fungicidally effective amount of a compound corresponding to any of the compound embodiments describe above. Of particular note are embodiments where the compounds are applied as compositions of this invention.

One or more of the following methods and variations as described in Schemes 1-17 can be used to prepare the compounds of Formula 1. The definitions of Q¹, Q², R¹, R², R³, and R⁴ in the compounds of Formulae 1-19 below are as defined above in the Summary of the Invention unless otherwise noted. Formulae 1a, 1b, 1c, and ld are various subsets of Formula 1. Formulae 4a and 4b are subsets of Formula 4. Substituents for each subset formula are as defined for its parent formula unless otherwise noted.

As illustrated in Scheme 1, compounds of Formula 1 wherein R³ and R⁴ are both halogens can be prepared by reacting keto pyrazoles of Formula 2 with appropriate reagents. Reaction of compounds of Formula 2 with diethylaminosulfur trifluoride (DAST) or [bis(2-methoxyethyl)amino]sulfur trifluoride (BAST) in a solvent such as dichloromethane at 25-90° C. for time periods of 1-24 h, using procedures such as described in Tetrahedron 2008, 64, 9837-9842, provides compounds of Formula 1 wherein R³ and R⁴ are both F. Compounds of Formula 2 can also be treated with thionyl chloride or phosphorus pentachloride in presence of a base such as triethylamine or pyridine in a solvent such as dichloromethane or N,N-dimethylformamide at 90-140° C. for time periods of 1-24 h, using procedures such as described in J. Med. Chem. 2008, 51, 2115-2127 and Tetrahedron Letters 2006, 48, 389-39, to provide compounds of Formula 1 wherein R³ and R⁴ are both Cl. Analogously by using thionyl bromide or phosphorus pentabromide, compounds of Formula 1 wherein R³ and R⁴ are both Br, can be isolated.

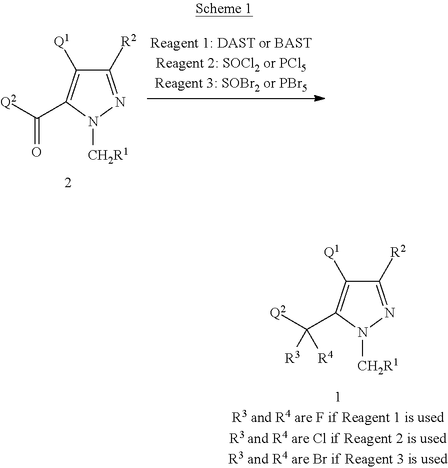

Compounds of Formula 1 wherein R³ and R⁴ are both I (iodine) can be prepared by conversion of ketones of Formula 2 to the corresponding hydrazones, which are then reacted with elemental iodine in the presence of a base such as triethylamine and a solvent such as ethyl ether, according to the general method described in Australian Journal of Chemistry 1970, 23, 989-1003.

Compounds of Formula 1 wherein R³ and R⁴ are different halogens can be prepared by variations of the method of Scheme 1 using appropriate mixtures of reagents to provide two different halogens in mixtures of products for separation. Furthermore, certain methods can provide particular combinations of halogens. For example, by general literature methods (see, e.g., Chem. Ber. 1954, 87, 1449-1460; Recueil des Travaux Chimiques des Pays-Bas 1971, 90, 866-873) ketones of Formula 2 can be converted to corresponding oximes and then chlorinated to provide gem-chloronitroso compounds (analogous to Formula 1 wherein one of R³ and R⁴ is Cl and the other of R³ and R⁴ is —NO), which are then treated with elemental bromine or iodine in the presence of light according to a literature method (Tetrahedron Letters 1976, 17, 943-944) to provide compounds of Formula 1 wherein one of R³ and R⁴ is Cl and the other of R³ and R⁴ is Br or I, respectively.

Alternatively methods are known in the art for replacing one halogen with another. For example as illustrated in Scheme 2, compounds of Formula 1b (i.e. Formula 1 wherein R³ is F and R⁴ is Cl) can be prepared by treating compounds of Formula 1a (i.e. Formula 1 wherein R³ and R⁴ are both Cl) with potassium fluoride or cesium fluoride in presence of a solvent such as dimethyl sulfoxide or N,N-dimethylformamide at 0-25° C. for time periods of 30 min to 4 h, using procedures such as described in Zhurnal Organicheskoi Khimii 1983, 19, 2164-73.

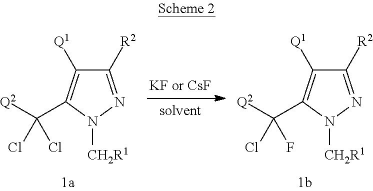

As illustrated in Scheme 3, compounds of Formula 1c (i.e. Formula 1, wherein R³ is an alkyl, and R⁴ is a halogen) can be prepared by treating compounds of Formula 3 with appropriate reagents as explained below.

Compounds of Formula 1c wherein R³ is alkyl, and R⁴ is F can be prepared by reacting compounds of Formula 3 with DAST or BAST in a solvent such as dichloromethane at 25-90° C. for time periods of 1-24 h, using procedures such as described in Heterocycles 2006, 67, 247-254. Compounds of Formula 3 can also be treated with thionyl chloride or phosphorus pentachloride in presence of a base such as triethylamine or pyridine in a solvent such as dichloromethane or N,N-dimethylformamide at 90-140° C. for time periods of 1-24 h, using procedures such as described in Bull. Chem. Soc. Japan 2002, 75, 1371-1379, to provide compounds of Formula 1c wherein R³ is alkyl, and R⁴ is Cl. Analogously by using thionyl bromide or phosphorus pentabromide, compounds of Formula 1c wherein R³ is alkyl, and R⁴ is Br can be isolated.

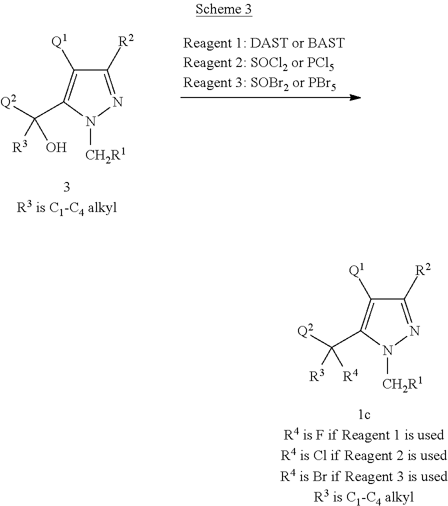

As shown in Scheme 4, compounds of Formula 3, can be prepared by treating keto compounds of Formula 2 with alkylmagnesium halides in presence of zinc chloride and a solvent such as diethyl ether or tetrahydrofuran at 0-90° C. for time periods of 30 min to 2 h, using procedures such as described in Organic Lett. 2009, 11, 1659-1662, J. Am. Chem. Soc. 2006, 128, 9998-9999, and Acta Chemica Scandinavica 1991, 45, 925-929.

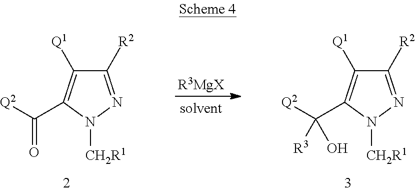

The keto intermediate of Formula 2 can be prepared from its hydroxylprecursor, as illustrated in Scheme 5, by reacting compounds of Formula 4 with an oxidizing agent such as pyridinium chlorochromate or manganese dioxide in a solvent such as dichloromethane, methanol or water at 20-100° C., according to general methods described in Tetrahedron Letters 2002, 43, 6149-6150.

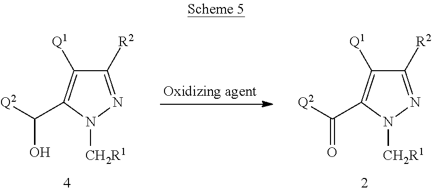

The hydroxyl intermediate of Formula 4 can further be used to prepare other embodiments of the invention.

As illustrated in Scheme 6, compounds of Formula 1d (i.e. Formula 1 in which R³ is H) can be prepared by general methods described in J. Org. Chem. 1982, 47, 5220-5222 and Eur. J. Med. Chem. 2009, 44, 1223-1229. Compounds of Formula 4 can be treated with DAST in a solvent such as dichloromethane or tetrahydrofuran at 0-60° C. to provide compounds of Formula 1d wherein R⁴ is F. Compounds of Formula 4 can also be treated with thionyl chloride or phosphorus pentachloride in presence of a base such as triethylamine or pyridine in a solvent such as dichloromethane or pyridine at 25-110° C. to provide a compound of Formula 1d wherein R⁴ is Cl. Analogously by using thionyl bromide or phosphorus pentabromide, compounds of Formula 1d wherein R⁴ is Br can be isolated. Compounds of Formula 1d wherein R⁴ is I can be prepared by reacting compounds of Formula 4 with sodium iodide or potassium iodide in presence of BF₃.Et₂O and an ether solvent such as 1,4-dioxane or with hydroiodic acid in a solvent such as acetonitrile at 25-70° C. for time periods of 15 min to 20 h, according to general methods described in Tetrahedron Letters 2001, 42, 951-953 and J. Am. Chem. Soc. 1965, 87, 539-42. The method of Scheme 6 using Reagent 1 (i.e. DAST) to prepare a compound of Formula 1d wherein R² is F is illustrated by Step E of Synthesis Example 1. The method of Scheme 6 using Reagent 2 (i.e. SOCl₂) to prepare a compound of Formula 1d wherein R² is Cl is illustrated by Synthesis Example 2.

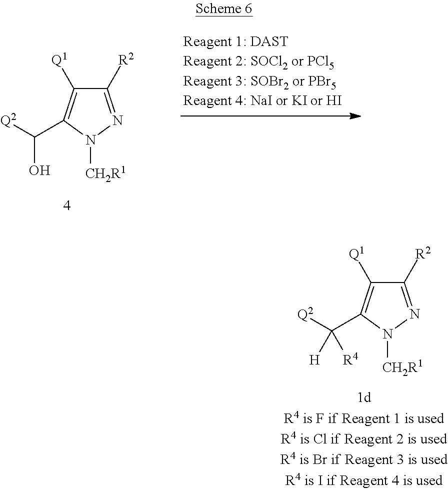

As shown in Scheme 7 compounds of Formula 4 can be prepared by treatment of compounds of Formula 5 with an organometallic reagent (6) such as an alkyllithium, preferably n-butyllithium, or an alkylmagnesium reagent, preferably isopropylmagnesium chloride (optionally complexed with lithium chloride) to form a metallated intermediate of Formula 7, followed by the addition of a carbonyl electrophile of Formula 8. Reaction temperatures can range from −90° C. to the boiling point of the reaction solvent; temperatures of −78° C. to ambient temperature are typical, with temperatures of −78 to −10° C. preferred when an alkyllithium reagent is used, and −20° C. to ambient temperature preferred with use of alkylmagnesium reagents. A variety of anhydrous solvents are useful, such as toluene, ethyl ether, tetrahydrofuran or dimethoxymethane. The Q²-containing carbonyl intermediates of Formula 8 are commercially available or can be prepared by methods known in the art.

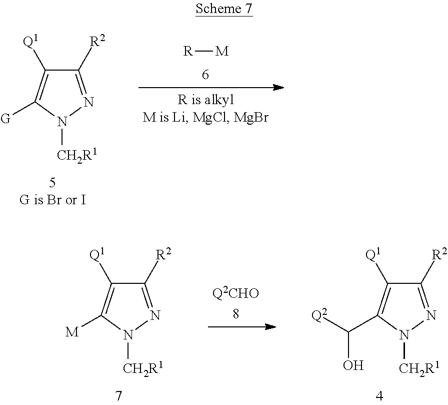

As shown in Scheme 8, compounds of Formula 5 wherein G is Br or I can be prepared by reaction of 5-aminopyrazoles of Formula 9 under diazotization conditions either in the presence of, or followed by combination with, copper salts containing bromide or iodide. For example, addition of tert-butyl nitrite to a solution of a 5-aminopyrazole of Formula 9 in the presence of CuBr₂ in a solvent such as acetonitrile provides the corresponding 5-bromopyrazole of Formula 5. Likewise, a 5-aminopyrazole of Formula 9 can be converted to a diazonium salt and then to a corresponding 5-halopyrazole of Formula 5 by treatment with sodium nitrite in a solvent such as water, acetic acid or trifluoroacetic acid, in the presence of a mineral acid typically containing the same halide atom (such as aqueous HI solution for G being I), followed by treatment with the corresponding copper(I) or copper(II) salt according to general procedures well known to those skilled in the art, such as described in Tetrahedron Lett. 2000, 41(24), 4713-4716. The method of Scheme 8 is illustrated by Step C of Synthesis Example 1.

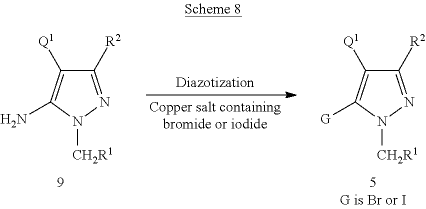

General methods useful for preparing 5-aminopyrazoles of Formula 9 are well known in the art; see, for example, Journal fir Praktische Chemie (Liepzig) 1911, 83, 171-182 and J. Am. Chem. Soc. 1954, 76, 501-503. In one method, as shown in Scheme 9, compounds of Formula 9 are prepared by treating compounds of Formula 10 with alkyl hydrazines of Formula 11, optionally in presence of an acidic catalyst such as acetic acid. The method of Scheme 9 is illustrated by Step B of Synthesis Example 1.

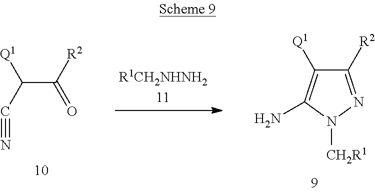

As illustrated in Scheme 10, compounds of Formula 10 can be prepared by reacting compounds of Formula 11 with optionally substituted alkanoic or cycloalkanoic acid ethyl esters of Formula 12, in presence of base such as sodium ethoxide, potassium t-butoxide or sodium hydride. Reaction temperatures can range from ambient temperatures (e.g., about 18 to 30° C.) to 100° C., for time periods of 10 min to 5 h. Typical solvents used are tetrahydrofuran or ethanol. See, for example, Bioorganic & Medicinal Chemistry 2006, 14, 1785-1791, Organic Syntheses Coll. Vol. 2, p. 487-489, and Organic Reactions, John Wiley & Sons, Inc. 1984, Vol. 31, pp. 31 and 38, and references cited therein. The method of Scheme 10 is illustrated by Step A of Synthesis Example 1.

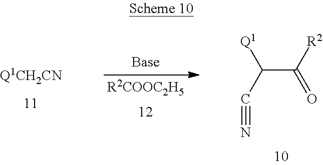

Alternatively, in order to introduce halogens at the 3-position of the pyrazole ring, compounds of Formula 4, wherein R² is fluorine, chlorine, bromine or iodine, can be made via different synthetic routes as explained in the schemes below.

As illustrated in Scheme 11, compounds of Formula 4, wherein R² is chlorine, bromine or iodine are prepared by treating compounds of Formula 13 with the corresponding N-halosuccinimide in presence of a suitable solvent such as N,N-dimethylformamide or tetrahydrofuran at 20 to 60° C. for a time period of 30 min to 15 h, according to general procedures known in the art such as described in Tetrahedron Lett. 2009, 50, 5762-5764.

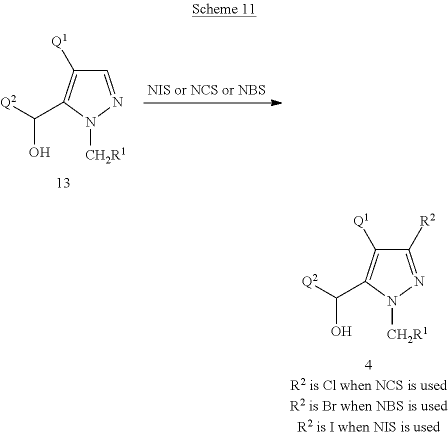

To introduce a fluoro at the 3-position of the pyrazole ring, compounds Formula 4a (i.e. Formula 4 wherein R² is chlorine) are treated under conditions similar to those employed for the method of Scheme 2, as shown in Scheme 12.

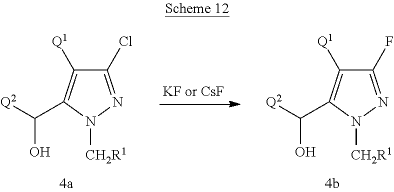

As shown in Scheme 13, compounds of Formula 13 can be prepared from compounds of Formula 14 under conditions similar to those employed for the method of Scheme 7.

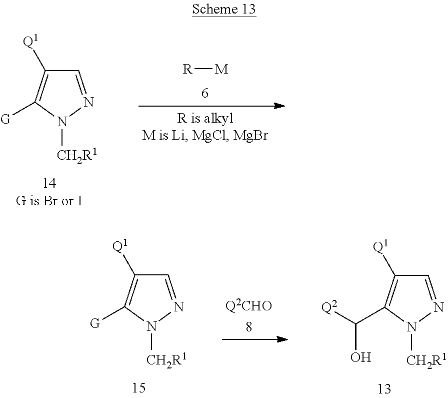

As shown in Scheme 14, compounds of Formula 14, can be prepared by treating compounds of Formula 16 with an alkyl iodide of Formula 17 in presence of a base such as sodium hydride or potassium carbonate in a solvent such as tetrahydrofuran or toluene at 0° C. to ambient temperatures, for time periods of 30 min to 15 h. See, for example, Synth. Commun. 2008, 38, 674-683, and PCT Patent Publication WO 2006/092510.

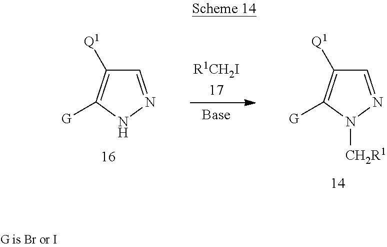

As illustrated in Scheme 15, compounds of Formula 16 are prepared from compounds of Formula 18 under conditions similar to those employed for the method of Scheme 8.

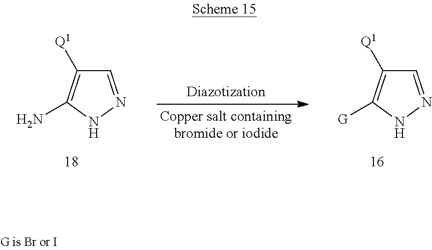

As illustrated in Scheme 16, compounds of Formula 18 can be prepared by treating compounds of Formula 19 with hydrazine hydrate in presence of an acid such as acetic acid in a suitable solvent such as toluene or N,N-dimethylformamide at temperatures ranging from ambient temperatures to 100° C. for time periods of 2 min to 16 h, such as described in J. Heterocyclic Chem. 2008, 45, 307-310, ARKIVOC 2006, (15), 133-141, and PCT Patent Publication 2007/147647.

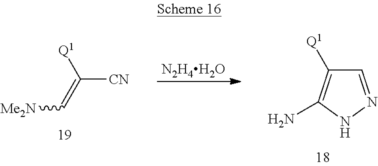

As shown in Scheme 17, compounds of Formula 19 can be prepared by reacting compounds of Formula 11 with dimethylformamide-dimethyl acetal in presence of a solvent such as toluene or xylene at temperatures ranging from ambient temperatures to 120° C. for time periods of 1 h to 3.5 h, such as described in J. Med. Chem. 2008, 51, 3777-3787, and PCT Patent Publication 2005/070431.

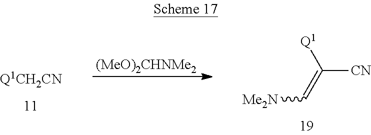

It is recognized by one skilled in the art that various functional groups can be converted into others to provide different compounds of Formula 1. For example, compounds of Formula 1 in which R² is methyl, ethyl or cyclopropyl can be modified by free-radical halogenation to form compounds of Formula 1 wherein R² is halomethyl, haloethyl or halocyclopropyl. The halomethyl compounds can be used as intermediates to prepare compounds of Formula 1 wherein R² is hydroxymethyl or cyanomethyl. Compounds of Formula 1 or intermediates for their preparation may contain aromatic nitro groups, which can be reduced to amino groups, and then be converted via reactions well known in the art such as the Sandmeyer reaction, to various halides, providing other compounds of Formula 1. By similar known reactions, aromatic amines (anilines) can be converted via diazonium salts to phenols, which can then be alkylated to prepare compounds of Formula 1 with alkoxy substituents. Likewise, aromatic halides such as bromides or iodides prepared via the Sandmeyer reaction can react with alcohols under copper-catalyzed conditions, such as the Ullmann reaction or known modifications thereof, to provide compounds of Formula 1 that contain alkoxy substituents. Additionally, some halogen groups, such as fluorine or chlorine, can be displaced with alcohols under basic conditions to provide compounds of Formula 1 containing the corresponding alkoxy substituents. The resultant alkoxy compounds can themselves be used in further reactions to prepare compounds of Formula 1 wherein R⁵ is -U-V-T (see, for example, PCT Publication WO 2007/149448 A2). Compounds of Formula 1 or precursors thereof in which R² or R³ is halide, preferably bromide or iodide, are particularly useful intermediates for transition metal-catalyzed cross-coupling reactions to prepare compounds of Formula 1. These types of reactions are well documented in the literature; see, for example, Tsuji in Transition Metal Reagents and Catalysts: Innovations in Organic Synthesis, John Wiley and Sons, Chichester, 2002; Tsuji in Palladium in Organic Synthesis, Springer, 2005; and Miyaura and Buchwald in Cross Coupling Reactions: A Practical Guide, 2002; and references cited therein.

One skilled in the art will recognize that sulfide groups can be oxidized to the corresponding sulfoxides or sulfones by conditions well-known in the art. The above reactions can also in many cases be performed in alternate sequence, such as the preparation of 1H pyrazoles for use in the reaction in Scheme 2 by reactions illustrated later for the general preparation of substituted pyrazoles. The presence of certain functional groups may not be compatible with all of these reaction conditions, and the use of protecting groups may be desirable for obtaining the desired products with improved yields and or purity.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1. One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane in CDCl₃; “s” means singlet, “m” means multiplet. Mass spectra are reported as the molecular weight of the highest isotopic abundance parent ion (M+1) formed by addition of H⁺ (molecular weight of 1) to the molecule, observed by mass spectrometry using atmospheric pressure chemical ionization (AP⁺) where “amu” stands for atomic mass units.

### Synthesis Example 1

### Preparation of 4-(2-Chloro-4-fluorophenyl)-5-[(2,4-difluorophenyl)fluoromethyl]-1,3-dimethyl-1H-pyrazole (Compound 1)

### Step A: Preparation of α-Acetyl-2-chloro-4-fluorobenzeneacetonitrile

Solid sodium ethoxide (2.7 g, 0.04 mmol) was stirred in a mixture of xylene (12 mL) and ethanol (2 mL) and heated to 50° C. A solution of 2-chloro-4-fluorobenzeneacetonitrile (commercially available) (4.0 g, 0.02 mmol) in ethyl acetate (10 mL) was added dropwise. The reaction mixture was heated at 50° C. for 4 h and then allowed to cool to ambient temperature. The reaction mixture was poured into water (50 mL) and extracted with ethyl acetate (15 mL). The aqueous phase was acidified with 3 N aqueous HCl to pH 4 and extracted with ethyl acetate (70 mL). The organic phase was washed with water (25 mL) and brine (25 mL), then dried over MgSO₄, and concentrated to leave the title compound as a semisolid (4.0 g).

¹H NMR δ 7.42 (m, 1H), 7.00 (m, 1H), 6.93 (m, 1H), 2.36 (s, 3H). MS: 212 amu (AP⁺).

### Step B: Preparation of 4-(2-chloro-4-fluorophenyl)-1,3-dimethyl-1H-pyrazol-5-amine

α-Acetyl-2-chloro-4-fluorobenzeneacetonitrile (i.e. the product of Step A) (1.7 g, 0.007 mol) and sodium acetate (2.18 g, 0.014 mol) were stirred in ethanol (17 mL). Methylhydrazine sulfate (1.4 mg, 1.19 mol) was added to this mixture. The reaction mixture was heated at reflux for 16 h, cooled, and then poured into water (100 mL). The resulting mixture was extracted with ethyl acetate (100 mL). The organic phase was washed with brine (50 mL), dried over MgSO₄, and concentrated to leave title compound as a pale yellow solid (1.77 g).

¹H NMR δ 7.21 (m, 2H), 7.03 (m, 1H), 3.68 (s, 3H), 2.08 (s, 3H). MS: 240 amu (AP⁺).

### Step C: Preparation of 5-Bromo-4-(2-chloro-4-fluorophenyl)-1,3-dimethyl-1H-pyrazole

Copper(II) bromide (3.94 g, 17.7 mmol) was added to a solution of 4-[2-chloro-4-fluorophenyl]-1,3-dimethyl-1H-pyrazol-5-amine (i.e. product of Step B) (2.4 g, 10 mmol) in acetonitrile (50 mL), and the mixture was stirred and cooled in an ice-water bath while tert-butyl nitrite (90% technical grade, 2.33 mL, 17.7 mmol) was added dropwise over 5 min. The reaction mixture was allowed to warm slowly to ambient temperature. Aqueous HCl solution (6 N solution, 20 mL) was added, and then ethyl acetate was added (20 mL). This mixture was filtered through a 2-cm pad of Celite® diatomaceous filter aid. The filter pad was washed with ethyl acetate (20 mL), and the phases were separated. The organic phase was washed with 1.0 N aqueous hydrochloric acid solution and brine, dried over MgSO₄, and concentrated to leave the title compound as an orange-brown semisolid (2.8 g).

¹H NMR δ 7.18-7.25 (m, 2H), 7.04 (m, 1H), 3.89 (s, 3H), 2.14 (s, 3H). MS: 369 amu (AP⁺).

### Step D: Preparation of 4-(2-Chloro-4-fluorophenyl)-α-(2,4-difluorophenyl)-1,3-dimethyl-1H-pyrazole-5-methanol

5-Bromo-4-(2-chloro-4-fluorophenyl)-1,3-dimethyl-1H-pyrazole (i.e. the product of Step C) (0.25 g, 0.82 mmol) was dissolved in anhydrous tetrahydrofuran (12 mL), and the mixture was cooled in a dry ice/acetone bath under a nitrogen atmosphere. A cyclohexane solution of n-butyllithium (2.0 M, 0.49 mL, 0.98 mmol) was added dropwise over 5 minutes. After 15 minutes, a solution of 2,4-difluorobenzaldehyde (0.09 mL, 0.82 mmol) in anhydrous tetrahydrofuran (3 mL) was added slowly dropwise, causing the dark red-colored solution to lighten to a yellow color. After 45 minutes, the reaction mixture was quenched by the addition of saturated aqueous NH₄Cl solution (˜20 mL) and allowed to warm to ambient temperature. This mixture was extracted with ethyl acetate, and the organic phase was washed with saturated aqueous NH₄Cl solution (25 mL) and with brine, dried over Na₂SO₄, and concentrated to leave a viscous residue. This residue was purified by column chromatography through silica gel eluted with a gradient of ethyl acetate in hexane (7% to 10%) to give the title compound as a white semi-solid (109 mg).

¹H NMR δ 7.5 (m, 1H), 7.1 (m, 2H), 7.0 (m, 1H), 6.85 (m, 2H), 5.9 (s, 1H), 3.8 (s, 3H), 2.1 (s, 3H). MS: 367 amu (AP⁺).

### Step E: Preparation of 4-(2-Chloro-4-fluorophenyl)-5-[(2,4-difluorophenyl)fluoromethyl]-1,3-dimethyl-1H-pyrazole

4-(2-Chloro-4-fluorophenyl)-α-(2,4-difluorophenyl)-1,3-dimethyl-1H-pyrazole-5-methanol (i.e. the product of Step D) (82 mg, 0.22 mmol) was dissolved in dichloromethane (5 mL), and the solution was stirred under a nitrogen atmosphere and cooled using an ice-water bath. (Diethylamino)sulfur trifluoride (54 mg, 0.33 mmol) was added dropwise, and the reaction mixture was allowed to warm slowly to ambient temperature over 1 h. The reaction mixture was partitioned between water (5 mL) and dichloromethane (5 mL). The organic phase was washed with additional water (5 mL) and with brine (5 mL), dried over Na₂SO₄ and concentrated under reduced pressure to give a viscous residue. The residue was purified by column chromatography through silica gel eluted with a gradient of ethyl acetate in hexane (16% to 20%) to give the title product, a compound of the present invention, as a viscous oil (44 mg).

¹H NMR δ 7.13 (m, 1H), 6.98 (m, 1H), 6.80-6.91 (m, 2H), 6.62 (m, 2H), 6.15 (m, 1H), 3.84 (s, 3H), 1.97 (s, 3H). MS: 369 amu (AP⁺).

### Synthesis Example 2

### Preparation of 5-[Chloro(2,4-difluorophenyl)methyl]-4-(2-chloro-4-fluorophenyl)-1,3-dimethyl-1H-pyrazole (Compound 2)

4-(2-Chloro-4-fluorophenyl)-α-(2,4-difluorophenyl)-1,3-dimethyl-1H-pyrazole-5-methanol (i.e. the product of Synthesis Example 1, Step D) (50 mg, 0.14 mmol) was dissolved in dichloromethane, and triethylamine (0.03 ml, 0.21 mmol) was added dropwise, followed by addition of thionyl chloride (0.01 mL, 0.17 mmol). The mixture was stirred at ambient temperature for 2 h, then concentrated and partitioned between dichloromethane (5 mL) and water (5 mL). The organic phase was washed with additional water (5 mL) and with brine (5 mL), dried over Na₂SO₄ and concentrated under reduced pressure to give a viscous residue. This residue was purified by column chromatography through silica gel eluted with ethyl acetate in hexane (10%) to give the title product, a compound of the present invention, as a white viscous oil (23 mg).

¹H NMR δ 7.83 (m, 1H), 7.30 (s, 1H), 6.86-6.93 (m, 3H), 6.59-6.71 (m, 1H), 3.83 (s, 3H), 2.11 (s, 3H). MS: 385 amu (AP⁺).

By the procedures described herein together with methods known in the art, the compounds disclosed in the Tables that follow can be prepared. The following abbreviations are used in the Tables which follow: Me means methyl, Et means ethyl, n-Pr means n-propyl, c-Pr means cyclopropyl, Ph means phenyl, Py means pyridinyl, Th means thienyl, MeO means methoxy, EtO means ethoxy, and —CN means cyano.

TABLE 1

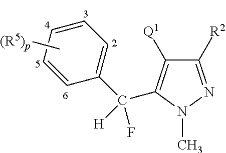

Q¹ is 2,6-di-F—Ph, and R² is Me.

| (R⁵)ₚ | (R⁵)ₚ |
|---|---|
| 2-F | 2-F-4-CN |
| 4-Cl | 2-Cl-4,6-di-F |
| 2,6-di-F | 4-Br-2,5-di-F |
| 2-Cl-4-F | 2-CF₃-4-F |
| 2-Br-4-F | 2-Br-4-Cl |
| 2-Br-4-MeO | 2-Cl |
| 2,6-di-F-4-CN | 4-Br |
| 4-Cl-2,5-di-F | 2,3,5-tri-F |
| 2,6-di-Cl-4-F | 2,6-di-Cl |
| 2-F-4-Br | 2-F-4-MeO |
| 3-F | 2-Cl-4-CN |
| 2-Br | 2-Br-4,5-di-F |
| 2,4,6-tri-F | 4-Br-2,6-di-F |
| 2-F-4-Cl | 4-Me |
| 2-I-4-F | 2-Br-4-F-6-Cl |
| 2,6-di-F-4-MeO | 3-Cl |
| 2-Cl-4,5-di-F | 2,4-di-F |
| 4-Cl-2,6-di-F | 2,3,6-tri-F |
| 2,6-di-Cl-4-MeO | 2,4,6-tri-Cl |
| 2-Cl-4-Br | 2-Cl-4-MeO |
| 4-F | 2-Br-4-CN |
| 3-Br | 2-Br-4,6-di-F |
| 2,4,5-tri-F | 2,4-di-Cl-6-F |
| 2,4-di-Cl | 2,4-di-Me |
| 2-Me-4-F | 2-Cl-4-Br-6-F |

The present disclosure also includes Tables 2 through 96, each of which is constructed the same as Table 1 above, except that the row heading in Table 1 (i.e. “Q¹ is 2,6-di-F-Ph, and R² is Me”) is replaced with the respective row heading shown below. For example, in Table 2 the row heading is “Q¹ is 2,6-di-F-Ph, and R² is Cl” and (R⁵)ₚ is as defined in Table 1 above. Thus, the first entry in Table 2 specifically discloses 3-chloro-4-(2,6-difluorophenyl)-5-[fluoro(2-fluorophenyl)methyl]-1-methyl-1H-pyrazole. Tables 3 through 96 are constructed similarly.

| Table | Row Heading |
|---|---|
| 2 | Q¹ is 2,6-di-F—Ph, and R² is Cl. |
| 3 | Q¹ is 2,6-di-F—Ph, and R² is Br. |
| 4 | Q¹ is 2,4-di-F—Ph, and R² is Me. |
| 5 | Q¹ is 2,4-di-F—Ph, and R² is Cl. |
| 6 | Q¹ is 2,4-di-F—Ph, and R² is Br. |
| 7 | Q¹ is 2,4,6-tri-F—Ph, and R² is Me. |
| 8 | Q¹ is 2,4,6-tri-F—Ph, and R² is Cl. |
| 9 | Q¹ is 2,4,6-tri-F—Ph, and R² is Br. |
| 10 | Q¹ is 2,6-di-F-4-MeO—Ph, and R² is Me. |
| 11 | Q¹ is 2,6-di-F-4-MeO—Ph, and R² is Cl. |
| 12 | Q¹ is 2,6-di-F-4-MeO—Ph, and R² is Br. |
| 13 | Q¹ is 2,6-di-F-4-EtO—Ph, and R² is Me. |
| 14 | Q¹ is 2,6-di-F-4-EtO—Ph, and R² is Cl. |
| 15 | Q¹ is 2,6-di-F-4-EtO—Ph, and R² is Br. |
| 16 | Q¹ is 2,6-di-F-4-CN—Ph, and R² is Me. |
| 17 | Q¹ is 2,6-di-F-4-CN—Ph, and R² is Cl. |
| 18 | Q¹ is 2,6-di-F-4-CN—Ph, and R² is Br. |
| 19 | Q¹ is 2-Cl-4-F—Ph, and R² is Me. |
| 20 | Q¹ is 2-Cl-4-F—Ph, and R² is Cl. |
| 21 | Q¹ is 2-Cl-4-F—Ph, and R² is Br. |
| 22 | Q¹ is 2-Cl-6-F—Ph, and R² is Me. |
| 23 | Q¹ is 2-Cl-6-F—Ph, and R² is Cl. |
| 24 | Q¹ is 2-Cl-6-F—Ph, and R² is Br. |
| 25 | Q¹ is 2-Cl-4,6-di-F—Ph, and R² is Me. |
| 26 | Q¹ is 2-Cl-4,6-di-F—Ph, and R² is Cl. |
| 27 | Q¹ is 2-Cl-4,6-di-F—Ph, and R² is Br. |
| 28 | Q¹ is 4-Cl-2,6-di-F—Ph, and R² is Me. |
| 29 | Q¹ is 4-Cl-2,6-di-F—Ph, and R² is Cl. |
| 30 | Q¹ is 4-Cl-2,6-di-F—Ph, and R² is Br. |
| 31 | Q¹ is 2-Br-4-F—Ph, and R² is Me. |
| 32 | Q¹ is 2-Br-4-F—Ph, and R² is Cl. |
| 33 | Q¹ is 2-Br-4-F—Ph, and R² is Br. |
| 34 | Q¹ is 2-Br-6-F—Ph, and R² is Me. |
| 35 | Q¹ is 2-Br-6-F—Ph, and R² is Cl. |
| 36 | Q¹ is 2-Br-6-F—Ph, and R² is Br. |
| 37 | Q¹ is 2-Me-4-F—Ph, and R² is Me. |
| 38 | Q¹ is 2-Me-4-F—Ph, and R² is Cl. |
| 39 | Q¹ is 2-Me-4-F—Ph, and R² is Br. |
| 40 | Q¹ is 2-I-4-F—Ph, and R² is Me. |
| 41 | Q¹ is 2-I-4-F—Ph, and R² is Cl. |
| 42 | Q¹ is 2-I-4-F—Ph, and R² is Br. |
| 43 | Q¹ is 2-F—Ph, and R² is Me. |
| 44 | Q¹ is 2-F—Ph, and R² is Cl. |
| 45 | Q¹ is 2-F—Ph, and R² is Br. |
| 46 | Q¹ is 2-Cl—Ph, and R² is Me. |
| 47 | Q¹ is 2-Cl—Ph, and R² is Cl. |
| 48 | Q¹ is 2-Cl—Ph, and R² is Br. |
| 49 | Q¹ is 2-Br—Ph, and R² is Me. |
| 50 | Q¹ is 2-Br—Ph, and R² is Cl. |
| 51 | Q¹ is 2-Br—Ph, and R² is Br. |
| 52 | Q¹ is 2-F-4-Cl—Ph, and R² is Me. |
| 53 | Q¹ is 2-F-4-Cl—Ph, and R² is Cl. |
| 54 | Q¹ is 2-F-4-Cl—Ph, and R² is Br. |
| 55 | Q¹ is 2,4-di-Cl—Ph, and R² is Me. |
| 56 | Q¹ is 2,4-di-Cl—Ph, and R² is Cl. |
| 57 | Q¹ is 2,4-di-Cl—Ph, and R² is Br. |
| 58 | Q¹ is 2,6-di-Cl—Ph, and R² is Me. |
| 59 | Q¹ is 2,6-di-Cl—Ph, and R² is Cl. |
| 60 | Q¹ is 2,6-di-Cl—Ph, and R² is Br. |
| 61 | Q¹ is 2-F-4-MeO—Ph, and R² is Me. |
| 62 | Q¹ is 2-F-4-MeO—Ph, and R² is Cl. |
| 63 | Q¹ is 2-F-4-MeO—Ph, and R² is Br. |
| 64 | Q¹ is 2-F-4-EtO—Ph, and R² is Me. |
| 65 | Q¹ is 2-F-4-EtO—Ph, and R² is Cl. |
| 66 | Q¹ is 2-F-4-EtO—Ph, and R² is Br. |
| 67 | Q¹ is 2-Cl-4-MeO—Ph, and R² is Me. |
| 68 | Q¹ is 2-Cl-4-MeO—Ph, and R² is Cl. |
| 69 | Q¹ is 2-Cl-4-MeO—Ph, and R² is Br. |
| 70 | Q¹ is 2-Cl-4-EtO—Ph, and R² is Me. |
| 71 | Q¹ is 2-Cl-4-EtO—Ph, and R² is Cl. |
| 72 | Q¹ is 2-Cl-4-EtO—Ph, and R² is Br. |
| 73 | Q¹ is 2-Br-4-MeO—Ph, and R² is Me. |
| 74 | Q¹ is 2-Br-4-MeO—Ph, and R² is Cl. |
| 75 | Q¹ is 2-Br-4-MeO—Ph, and R² is Br. |
| 76 | Q¹ is 2-Br-4-EtO—Ph, and R² is Me. |
| 77 | Q¹ is 2-Br-4-EtO—Ph, and R² is Cl. |
| 78 | Q¹ is 2-Br-4-EtO—Ph, and R² is Br. |
| 79 | Q¹ is 2-F-4-CN—Ph, and R² is Me. |
| 80 | Q¹ is 2-F-4-CN—Ph, and R² is Cl. |
| 81 | Q¹ is 2-F-4-CN—Ph, and R² is Br. |
| 82 | Q¹ is 2-Cl-4-CN—Ph, and R² is Me. |
| 83 | Q¹ is 2-Cl-4-CN—Ph, and R² is Cl. |
| 84 | Q¹ is 2-Cl-4-CN—Ph, and R² is Br. |
| 85 | Q¹ is 2-Br-4-CN—Ph, and R² is Me. |
| 86 | Q¹ is 2-Br-4-CN—Ph, and R² is Cl. |
| 87 | Q¹ is 2-Br-4-CN—Ph, and R² is Br. |
| 88 | Q¹ is 2,5-di-Cl-3-Py, and R² is Me. |
| 89 | Q¹ is 2,5-di-Cl-3-Py, and R² is Cl. |
| 90 | Q¹ is 2,5-di-Cl-3-Py, and R² is Br. |
| 91 | Q¹ is 2-Cl-3-Th, and R² is Me. |
| 92 | Q¹ is 2-Cl-3-Th, and R² is Cl. |
| 93 | Q¹ is 2-Cl-3-Th, and R² is Br. |
| 94 | Q¹ is 2,5-di-Cl-3-Th, and R² is Me. |
| 95 | Q¹ is 2,5-di-Cl-3-Th, and R² is Cl. |
| 96 | Q¹ is 2,5-di-Cl-3-Th, and R² is Br. |

TABLE 97

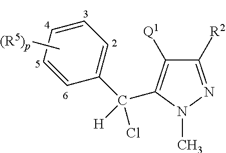

Q¹ is 2,6-di-F—Ph, and R² is Me.

| (R⁵)ₚ | (R⁵)ₚ |
|---|---|
| 2-F | 2-F-4-CN |
| 4-Cl | 2-Cl-4,6-di-F |
| 2,6-di-F | 4-Br-2,5-di-F |
| 2-Cl-4-F | 2-CF₃-4-F |
| 2-Br-4-F | 2-Br-4-Cl |
| 2-Br-4-MeO | 2-Cl |
| 2,6-di-F-4-CN | 4-Br |
| 4-Cl-2,5-di-F | 2,3,5-tri-F |
| 2,6-di-Cl-4-F | 2,6-di-Cl |
| 2-F-4-Br | 2-F-4-MeO |
| 3-F | 2-Cl-4-CN |
| 2-Br | 2-Br-4,5-di-F |
| 2,4,6-tri-F | 4-Br-2,6-di-F |
| 2-F-4-Cl | 4-Me |
| 2-I-4-F | 2-Br-4-F-6-Cl |
| 2,6-di-F-4-MeO | 3-Cl |
| 2-Cl-4,5-di-F | 2,4-di-F |
| 4-Cl-2,6-di-F | 2,3,6-tri-F |
| 2,6-di-Cl-4-MeO | 2,4,6-tri-Cl |
| 2-Cl-4-Br | 2-Cl-4-MeO |
| 4-F | 2-Br-4-CN |
| 3-Br | 2-Br-4,6-di-F |
| 2,4,5-tri-F | 2,4-di-Cl-6-F |
| 2,4-di-Cl | 2,4-di-Me |
| 2-Me-4-F | 2-Cl-4-Br-6-F |

The present disclosure also includes Tables 98 through 192, each of which is constructed the same as Table 97 above, except that the row heading in Table 97 (i.e. “Q¹ is 2,6-di-F-Ph, and R² is Me”) is replaced with the respective row heading shown below. For example, in Table 98 the row heading is “Q¹ is 2,6-di-F-Ph, and R² is Cl”, and (R⁵)ₚ is as defined in Table 97 above. Thus, the first entry in Table 98 specifically discloses 3-chloro-5-[chloro(2-fluorophenyl)methyl]-4-(2,6-difluorophenyl)-1-methyl-1H-pyrazole.

| Table | Row Heading |
|---|---|
| 98 | Q¹ is 2,6-di-F—Ph, and R² is Cl. |
| 99 | Q¹ is 2,6-di-F—Ph, and R² is Br. |
| 100 | Q¹ is 2,4-di-F—Ph, and R² is Me. |
| 101 | Q¹ is 2,4-di-F—Ph, and R² is Cl. |
| 102 | Q¹ is 2,4-di-F—Ph, and R² is Br. |
| 103 | Q¹ is 2,4,6-tri-F—Ph, and R² is Me. |
| 104 | Q¹ is 2,4,6-tri-F—Ph, and R² is Cl. |
| 105 | Q¹ is 2,4,6-tri-F—Ph, and R² is Br. |
| 106 | Q¹ is 2,6-di-F-4-MeO—Ph, and R² is Me. |
| 107 | Q¹ is 2,6-di-F-4-MeO—Ph, and R² is Cl. |
| 108 | Q¹ is 2,6-di-F-4-MeO—Ph, and R² is Br. |
| 109 | Q¹ is 2,6-di-F-4-EtO—Ph, and R² is Me. |
| 110 | Q¹ is 2,6-di-F-4-EtO—Ph, and R² is Cl. |
| 111 | Q¹ is 2,6-di-F-4-EtO—Ph, and R² is Br. |
| 112 | Q¹ is 2,6-di-F-4-CN—Ph, and R² is Me. |
| 113 | Q¹ is 2,6-di-F-4-CN—Ph, and R² is Cl. |
| 114 | Q¹ is 2,6-di-F-4-CN—Ph, and R² is Br. |
| 115 | Q¹ is 2-Cl-4-F—Ph, and R² is Me. |
| 116 | Q¹ is 2-Cl-4-F—Ph, and R² is Cl. |
| 117 | Q¹ is 2-Cl-4-F—Ph, and R² is Br. |
| 118 | Q¹ is 2-Cl-6-F—Ph, and R² is Me. |
| 119 | Q¹ is 2-Cl-6-F—Ph, and R² is Cl. |
| 120 | Q¹ is 2-Cl-6-F—Ph, and R² is Br. |
| 121 | Q¹ is 2-Cl-4,6-di-F—Ph, and R² is Me. |
| 122 | Q¹ is 2-Cl-4,6-di-F—Ph, and R² is Cl. |
| 123 | Q¹ is 2-Cl-4,6-di-F—Ph, and R² is Br. |
| 124 | Q¹ is 4-Cl-2,6-di-F—Ph, and R² is Me. |
| 125 | Q¹ is 4-Cl-2,6-di-F—Ph, and R² is Cl. |
| 126 | Q¹ is 4-Cl-2,6-di-F—Ph, and R² is Br. |
| 127 | Q¹ is 2-Br-4-F—Ph, and R² is Me. |
| 128 | Q¹ is 2-Br-4-F—Ph, and R² is Cl. |
| 129 | Q¹ is 2-Br-4-F—Ph, and R² is Br. |
| 130 | Q¹ is 2-Br-6-F—Ph, and R² is Me. |
| 131 | Q¹ is 2-Br-6-F—Ph, and R² is Cl. |
| 132 | Q¹ is 2-Br-6-F—Ph, and R² is Br. |
| 133 | Q¹ is 2-Me-4-F—Ph, and R² is Me. |
| 134 | Q¹ is 2-Me-4-F—Ph, and R² is Cl. |
| 135 | Q¹ is 2-Me-4-F—Ph, and R² is Br. |
| 136 | Q¹ is 2-I-4-F—Ph, and R² is Me. |
| 137 | Q¹ is 2-I-4-F—Ph, and R² is Cl. |
| 138 | Q¹ is 2-I-4-F—Ph, and R² is Br. |
| 139 | Q¹ is 2-F—Ph, and R² is Me. |
| 140 | Q¹ is 2-F—Ph, and R² is Cl. |
| 141 | Q¹ is 2-F—Ph, and R² is Br. |
| 142 | Q¹ is 2-Cl—Ph, and R² is Me. |
| 143 | Q¹ is 2-Cl—Ph, and R² is Cl. |
| 144 | Q¹ is 2-Cl—Ph, and R² is Br. |
| 145 | Q¹ is 2-Br—Ph, and R² is Me. |
| 146 | Q¹ is 2-Br—Ph, and R² is Cl. |
| 147 | Q¹ is 2-Br—Ph, and R² is Br. |
| 148 | Q¹ is 2-F-4-Cl—Ph, and R² is Me. |
| 149 | Q¹ is 2-F-4-Cl—Ph, and R² is Cl. |
| 150 | Q¹ is 2-F-4-Cl—Ph, and R² is Br. |
| 151 | Q¹ is 2,4-di-Cl—Ph, and R² is Me. |
| 152 | Q¹ is 2,4-di-Cl—Ph, and R² is Cl. |
| 153 | Q¹ is 2,4-di-Cl—Ph, and R² is Br. |
| 154 | Q¹ is 2,6-di-Cl—Ph, and R² is Me. |
| 155 | Q¹ is 2,6-di-Cl—Ph, and R² is Cl. |
| 156 | Q¹ is 2,6-di-Cl—Ph, and R² is Br. |
| 157 | Q¹ is 2-F-4-MeO—Ph, and R² is Me. |
| 158 | Q¹ is 2-F-4-MeO—Ph, and R² is Cl. |
| 159 | Q¹ is 2-F-4-MeO—Ph, and R² is Br. |
| 160 | Q¹ is 2-F-4-EtO—Ph, and R² is Me. |
| 161 | Q¹ is 2-F-4-EtO—Ph, and R² is Cl. |
| 162 | Q¹ is 2-F-4-EtO—Ph, and R² is Br. |
| 163 | Q¹ is 2-Cl-4-MeO—Ph, and R² is Me. |
| 164 | Q¹ is 2-Cl-4-MeO—Ph, and R² is Cl. |
| 165 | Q¹ is 2-Cl-4-MeO—Ph, and R² is Br. |
| 166 | Q¹ is 2-Cl-4-EtO—Ph, and R² is Me. |
| 167 | Q¹ is 2-Cl-4-EtO—Ph, and R² is Cl. |
| 168 | Q¹ is 2-Cl-4-EtO—Ph, and R² is Br. |
| 169 | Q¹ is 2-Br-4-MeO—Ph, and R² is Me. |
| 170 | Q¹ is 2-Br-4-MeO—Ph, and R² is Cl. |
| 171 | Q¹ is 2-Br-4-MeO—Ph, and R² is Br. |
| 172 | Q¹ is 2-Br-4-EtO—Ph, and R² is Me. |
| 173 | Q¹ is 2-Br-4-EtO—Ph, and R² is Cl. |
| 174 | Q¹ is 2-Br-4-EtO—Ph, and R² is Br. |
| 175 | Q¹ is 2-F-4-CN—Ph, and R² is Me. |
| 176 | Q¹ is 2-F-4-CN—Ph, and R² is Cl. |
| 177 | Q¹ is 2-F-4-CN—Ph, and R² is Br. |
| 178 | Q¹ is 2-Cl-4-CN—Ph, and R² is Me. |
| 179 | Q¹ is 2-Cl-4-CN—Ph, and R² is Cl. |
| 180 | Q¹ is 2-Cl-4-CN—Ph, and R² is Br. |
| 181 | Q¹ is 2-Br-4-CN—Ph, and R² is Me. |
| 182 | Q¹ is 2-Br-4-CN—Ph, and R² is Cl. |
| 183 | Q¹ is 2-Br-4-CN—Ph, and R² is Br. |
| 184 | Q¹ is 2,5-di-Cl-3-Py, and R² is Me. |
| 185 | Q¹ is 2,5-di-Cl-3-Py, and R² is Cl. |
| 186 | Q¹ is 2,5-di-Cl-3-Py, and R² is Br. |
| 187 | Q¹ is 2-Cl-3-Th, and R² is Me. |
| 188 | Q¹ is 2-Cl-3-Th, and R² is Cl. |
| 189 | Q¹ is 2-Cl-3-Th, and R² is Br. |
| 190 | Q¹ is 2,5-di-Cl-3-Th, and R² is Me. |
| 191 | Q¹ is 2,5-di-Cl-3-Th, and R² is Cl. |
| 192 | Q¹ is 2,5-di-Cl-3-Th, and R² is Br. |

TABLE 193

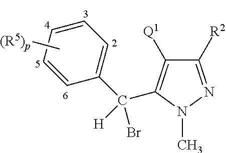

Q¹ is 2,6-di-F—Ph, and R² is Me.

| (R⁵)ₚ | (R⁵)ₚ |
|---|---|
| 2-F | 2-F-4-CN |
| 4-Cl | 2-Cl-4,6-di-F |
| 2,6-di-F | 4-Br-2,5-di-F |
| 2-Cl-4-F | 2-CF₃-4-F |
| 2-Br-4-F | 2-Br-4-Cl |
| 2-Br-4-MeO | 2-Cl |
| 2,6-di-F-4-CN | 4-Br |
| 4-Cl-2,5-di-F | 2,3,5-tri-F |
| 2,6-di-Cl-4-F | 2,6-di-Cl |
| 2-F-4-Br | 2-F-4-MeO |
| 3-F | 2-Cl-4-CN |
| 2-Br | 2-Br-4,5-di-F |
| 2,4,6-tri-F | 4-Br-2,6-di-F |
| 2-F-4-Cl | 4-Me |
| 2-I-4-F | 2-Br-4-F-6-Cl |
| 2,6-di-F-4-MeO | 3-Cl |
| 2-Cl-4,5-di-F | 2,4-di-F |
| 4-Cl-2,6-di-F | 2,3,6-tri-F |
| 2,6-di-Cl-4-MeO | 2,4,6-tri-Cl |
| 2-Cl-4-Br | 2-Cl-4-MeO |
| 4-F | 2-Br-4-CN |
| 3-Br | 2-Br-4,6-di-F |
| 2,4,5-tri-F | 2,4-di-Cl-6-F |
| 2,4-di-Cl | 2,4-di-Me |
| 2-Me-4-F | 2-Cl-4-Br-6-F |

The present disclosure also includes Tables 194 through 288, each of which is constructed the same as Table 193 above, except that the row heading in Table 193 (i.e. “Q¹ is 2,6-di-F-Ph, and R² is Me”) is replaced with the respective row heading shown below. For example, in Table 194 the row heading is “Q¹ is 2,6-di-F-Ph, and R² is Cl”, and (R⁵)ₚ is as defined in Table 193 above. Thus, the first entry in Table 194 specifically discloses 5-[bromo(2-fluorophenyl)methyl)-3-chloro-4-(2,6-difluorophenyl)-1-methyl-1H-pyrazole. Tables 195 through 288 are constructed similarly.

| Table | Row Heading |
|---|---|
| 194 | Q¹ is 2,6-di-F—Ph, and R² is Cl. |
| 195 | Q¹ is 2,6-di-F—Ph, and R² is Br. |
| 196 | Q¹ is 2,4-di-F—Ph, and R² is Me. |
| 197 | Q¹ is 2,4-di-F—Ph, and R² is Cl. |
| 198 | Q¹ is 2,4-di-F—Ph, and R² is Br. |
| 199 | Q¹ is 2,4,6-tri-F—Ph, and R² is Me. |
| 200 | Q¹ is 2,4,6-tri-F—Ph, and R² is Cl. |
| 201 | Q¹ is 2,4,6-tri-F—Ph, and R² is Br. |
| 202 | Q¹ is 2,6-di-F-4-MeO—Ph, and R² is Me. |
| 203 | Q¹ is 2,6-di-F-4-MeO—Ph, and R² is Cl. |
| 204 | Q¹ is 2,6-di-F-4-MeO—Ph, and R² is Br. |
| 205 | Q¹ is 2,6-di-F-4-EtO—Ph, and R² is Me. |
| 206 | Q¹ is 2,6-di-F-4-EtO—Ph, and R² is Cl. |
| 207 | Q¹ is 2,6-di-F-4-EtO—Ph, and R² is Br. |
| 208 | Q¹ is 2,6-di-F-4-CN—Ph, and R² is Me. |
| 209 | Q¹ is 2,6-di-F-4-CN—Ph, and R² is Cl. |
| 210 | Q¹ is 2,6-di-F-4-CN—Ph, and R² is Br. |
| 211 | Q¹ is 2-Cl-4-F—Ph, and R² is Me. |
| 212 | Q¹ is 2-Cl-4-F—Ph, and R² is Cl. |
| 213 | Q¹ is 2-Cl-4-F—Ph, and R² is Br. |
| 214 | Q¹ is 2-Cl-6-F—Ph, and R² is Me. |
| 215 | Q¹ is 2-Cl-6-F—Ph, and R² is Cl. |
| 216 | Q¹ is 2-Cl-6-F—Ph, and R² is Br. |
| 217 | Q¹ is 2-Cl-4,6-di-F—Ph, and R² is Me. |
| 218 | Q¹ is 2-Cl-4,6-di-F—Ph, and R² is Cl. |
| 219 | Q¹ is 2-Cl-4,6-di-F—Ph, and R² is Br. |
| 220 | Q¹ is 4-Cl-2,6-di-F—Ph, and R² is Me. |
| 221 | Q¹ is 4-Cl-2,6-di-F—Ph, and R² is Cl. |
| 222 | Q¹ is 4-Cl-2,6-di-F—Ph, and R² is Br. |
| 223 | Q¹ is 2-Br-4-F—Ph, and R² is Me. |
| 224 | Q¹ is 2-Br-4-F—Ph, and R² is Cl. |
| 225 | Q¹ is 2-Br-4-F—Ph, and R² is Br. |
| 226 | Q¹ is 2-Br-6-F—Ph, and R² is Me. |
| 227 | Q¹ is 2-Br-6-F—Ph, and R² is Cl. |
| 228 | Q¹ is 2-Br-6-F—Ph, and R² is Br. |
| 229 | Q¹ is 2-Me-4-F—Ph, and R² is Me. |
| 230 | Q¹ is 2-Me-4-F—Ph, and R² is Cl. |
| 231 | Q¹ is 2-Me-4-F—Ph, and R² is Br. |
| 232 | Q¹ is 2-I-4-F—Ph, and R² is Me. |
| 233 | Q¹ is 2-I-4-F—Ph, and R² is Cl. |
| 234 | Q¹ is 2-I-4-F—Ph, and R² is Br. |
| 235 | Q¹ is 2-F—Ph, and R² is Me. |
| 236 | Q¹ is 2-F—Ph, and R² is Cl. |
| 237 | Q¹ is 2-F—Ph, and R² is Br. |
| 238 | Q¹ is 2-Cl—Ph, and R² is Me. |
| 239 | Q¹ is 2-Cl—Ph, and R² is Cl. |
| 240 | Q¹ is 2-Cl—Ph, and R² is Br. |
| 241 | Q¹ is 2-Br—Ph, and R² is Me. |
| 242 | Q¹ is 2-Br—Ph, and R² is Cl. |
| 243 | Q¹ is 2-Br—Ph, and R² is Br. |
| 244 | Q¹ is 2-F-4-Cl—Ph, and R² is Me. |
| 245 | Q¹ is 2-F-4-Cl—Ph, and R² is Cl. |
| 246 | Q¹ is 2-F-4-Cl—Ph, and R² is Br. |
| 247 | Q¹ is 2,4-di-Cl—Ph, and R² is Me. |
| 248 | Q¹ is 2,4-di-Cl—Ph, and R² is Cl. |
| 249 | Q¹ is 2,4-di-Cl—Ph, and R² is Br. |
| 250 | Q¹ is 2,6-di-Cl—Ph, and R² is Me. |
| 251 | Q¹ is 2,6-di-Cl—Ph, and R² is Cl. |
| 252 | Q¹ is 2,6-di-Cl—Ph, and R² is Br. |
| 253 | Q¹ is 2-F-4-MeO—Ph, and R² is Me. |
| 254 | Q¹ is 2-F-4-MeO—Ph, and R² is Cl. |
| 255 | Q¹ is 2-F-4-MeO—Ph, and R² is Br. |
| 256 | Q¹ is 2-F-4-EtO—Ph, and R² is Me. |
| 257 | Q¹ is 2-F-4-EtO—Ph, and R² is Cl. |
| 258 | Q¹ is 2-F-4-EtO—Ph, and R² is Br. |
| 259 | Q¹ is 2-Cl-4-MeO—Ph, and R² is Me. |
| 260 | Q¹ is 2-Cl-4-MeO—Ph, and R² is Cl. |
| 261 | Q¹ is 2-Cl-4-MeO—Ph, and R² is Br. |
| 262 | Q¹ is 2-Cl-4-EtO—Ph, and R² is Me. |
| 263 | Q¹ is 2-Cl-4-EtO—Ph, and R² is Cl. |
| 264 | Q¹ is 2-Cl-4-EtO—Ph, and R² is Br. |
| 265 | Q¹ is 2-Br-4-MeO—Ph, and R² is Me. |
| 266 | Q¹ is 2-Br-4-MeO—Ph, and R² is Cl. |
| 267 | Q¹ is 2-Br-4-MeO—Ph, and R² is Br. |
| 268 | Q¹ is 2-Br-4-EtO—Ph, and R² is Me. |
| 269 | Q¹ is 2-Br-4-EtO—Ph, and R² is Cl. |
| 270 | Q¹ is 2-Br-4-EtO—Ph, and R² is Br. |
| 271 | Q¹ is 2-F-4-CN—Ph, and R² is Me. |
| 272 | Q¹ is 2-F-4-CN—Ph, and R² is Cl. |
| 273 | Q¹ is 2-F-4-CN—Ph, and R² is Br. |
| 274 | Q¹ is 2-Cl-4-CN—Ph, and R² is Me. |
| 275 | Q¹ is 2-Cl-4-CN—Ph, and R² is Cl. |
| 276 | Q¹ is 2-Cl-4-CN—Ph, and R² is Br. |
| 277 | Q¹ is 2-Br-4-CN—Ph, and R² is Me. |
| 278 | Q¹ is 2-Br-4-CN—Ph, and R² is Cl. |
| 279 | Q¹ is 2-Br-4-CN—Ph, and R² is Br. |
| 280 | Q¹ is 2,5-di-Cl-3-Py, and R² is Me. |
| 281 | Q¹ is 2,5-di-Cl-3-Py, and R² is Cl. |
| 282 | Q¹ is 2,5-di-Cl-3-Py, and R² is Br. |
| 283 | Q¹ is 2-Cl-3-Th, and R² is Me. |
| 284 | Q¹ is 2-Cl-3-Th, and R² is Cl. |
| 285 | Q¹ is 2-Cl-3-Th, and R² is Br. |
| 286 | Q¹ is 2,5-di-Cl-3-Th, and R² is Me. |
| 287 | Q¹ is 2,5-di-Cl-3-Th, and R² is Cl. |
| 288 | Q¹ is 2,5-di-Cl-3-Th, and R² is Br. |

TABLE 289

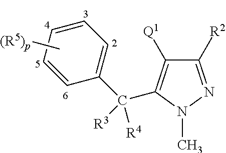

Q¹ is 2,6-di-F—Ph, R² is Me, R³ and R⁴ are both F.

| (R⁵)ₚ | (R⁵)ₚ |
|---|---|
| 2-F | 2-F-4-CN |
| 4-Cl | 2-Cl-4,6-di-F |
| 2,6-di-F | 4-Br-2,5-di-F |
| 2-Cl-4-F | 2-CF₃-4-F |
| 2-Br-4-F | 2-Br-4-Cl |
| 2-Br-4-MeO | 2-Cl |
| 2,6-di-F-4-CN | 4-Br |
| 4-Cl-2,5-di-F | 2,3,5-tri-F |
| 2,6-di-Cl-4-F | 2,6-di-Cl |
| 2-F-4-Br | 2-F-4-MeO |
| 3-F | 2-Cl-4-CN |
| 2-Br | 2-Br-4,5-di-F |
| 2,4,6-tri-F | 4-Br-2,6-di-F |
| 2-F-4-Cl | 4-Me |
| 2-I-4-F | 2-Br-4-F-6-Cl |
| 2,6-di-F-4-MeO | 3-Cl |
| 2-Cl-4,5-di-F | 2,4-di-F |
| 4-Cl-2,6-di-F | 2,3,6-tri-F |
| 2,6-di-Cl-4-MeO | 2,4,6-tri-Cl |
| 2-Cl-4-Br | 2-Cl-4-MeO |
| 4-F | 2-Br-4-CN |
| 3-Br | 2-Br-4,6-di-F |
| 2,4,5-tri-F | 2,4-di-Cl-6-F |
| 2,4-di-Cl | 2,4-di-Me |
| 2-Me-4-F | 2-Cl-4-Br-6-F |

The present disclosure also includes Tables 290 through 378, each of which is constructed the same as Table 289 above, except that the row heading in Table 289 (i.e. “Q¹ is 2,6-di-F-Ph, R² is Me, R³ and R⁴ are both F.”) is replaced with the respective row heading shown below. For example, in Table 290 the row heading is “Q¹ is 2,6-di-F-Ph, R² is Cl, R³ and R⁴ are both Cl”, and (R⁵)ₚ is as defined in Table 289 above. Thus, the first entry in Table 290 specifically discloses 3-chloro-5-[dichloro(2-fluorophenyl)methyl]-4-(2,6-difluorophenyl)-1-methyl-1H-pyrazole. Tables 291 through 378 are constructed similarly.

| Table | Row Heading |
|---|---|
| 290 | Q¹ is 2,6-di-F—Ph, R² is Cl, R³ and R⁴ are both Cl. |
| 291 | Q¹ is 2,6-di-F—Ph, R² is Br, R³ and R⁴ are both Br. |
| 292 | Q¹ is 2,6-di-F—Ph, R² is Br, R³ and R⁴ are both I. |
| 293 | Q¹ is 2,4-di-F—Ph, R² is Me, R³ is F, and R⁴ is Cl. |
| 294 | Q¹ is 2,4-di-F—Ph, R² is Br, R³ and R⁴ are both F. |
| 295 | Q¹ is 2,4,6-tri-F—Ph, R² is Me, R³ and R⁴ are both Cl. |
| 296 | Q¹ is 2,4,6-tri-F—Ph, R² is Cl, R³ and R⁴ are both Br. |
| 297 | Q¹ is 2,4,6-tri-F—Ph, R² is Br, R³ is F, and R⁴ is Cl. |
| 298 | Q¹ is 2-F—Ph, R² is Cl, R³ and R⁴ are both F. |
| 299 | Q¹ is 2-F—Ph, R² is Br, R³ and R⁴ are both Cl. |
| 300 | Q¹ is 2-Cl—Ph, R² is Me, R³ and R⁴ are both Br. |
| 301 | Q¹ is 2-Cl—Ph, R² is Cl, R³ is F, and R⁴ is Cl. |
| 302 | Q¹ is 2-Br—Ph, R² is Me, R³ and R⁴ are both F. |
| 303 | Q¹ is 2-Br—Ph, R² is Cl, R³ and R⁴ are both Cl. |
| 304 | Q¹ is 2-Br—Ph, R² is Br, R³ and R⁴ are both Br. |
| 305 | Q¹ is 2-F-4-Cl—Ph, R² is Me, R³ is F, and R⁴ is Cl. |
| 306 | Q¹ is 2-F-4-Cl—Ph, R² is Br, R³ and R⁴ are both F. |
| 307 | Q¹ is 2-Br-4-F—Ph, R² is Me, R³ and R⁴ are both Cl. |
| 308 | Q¹ is 2-Br-4-F—Ph, R² is Cl, R³ and R⁴ are both Br. |
| 309 | Q¹ is 2-Br-4-Cl—Ph, R² is Br, R³ is F, and R⁴ is Cl. |
| 310 | Q¹ is 2-Br-6-F—Ph, R² is Cl, R³ and R⁴ are both F. |
| 311 | Q¹ is 2-Br-6-F—Ph, R² is Br, R³ and R⁴ are both Cl. |
| 312 | Q¹ is 2-Cl-4-F—Ph, R² is Me, R³ and R⁴ are both Br. |
| 313 | Q¹ is 2-Cl-4-F—Ph, R² is Cl, R³ is F, and R⁴ is Cl. |
| 314 | Q¹ is 2-Cl-6-F—Ph, R² is Me, R³ and R⁴ are both F. |
| 315 | Q¹ is 2-Cl-6-F—Ph, R² is Cl, R³ and R⁴ are both Cl. |
| 316 | Q¹ is 2-Cl-6-F—Ph, R² is Br, R³ is F, and R⁴ is Cl. |
| 317 | Q¹ is 2,4-di-Cl—Ph, R² is Cl, R³ and R⁴ are both F. |
| 318 | Q¹ is 2,4-di-Cl—Ph, R² is Br, R³ and R⁴ are both Cl. |
| 319 | Q¹ is 2-I-4-F—Ph, R² is Me, R³ and R⁴ are both Br. |
| 320 | Q¹ is 2-I-4-F—Ph, R² is Cl, R³ is F, and R⁴ is Cl. |
| 321 | Q¹ is 2-I-4-F—Ph, R² is Br, R³ is F, and R⁴ is Cl. |
| 322 | Q¹ is 2-Me-4-F—Ph, R² is Cl, R³ and R⁴ are both F. |
| 323 | Q¹ is 2-Me-4-F—Ph, R² is Br, R³ and R⁴ are both Cl. |
| 324 | Q¹ is 2,6-di-Cl—Ph, R² is Me, R³ and R⁴ are both Br. |
| 325 | Q¹ is 2,6-di-Cl—Ph, R² is Cl R³ is F, and R⁴ is Cl. |
| 326 | Q¹ is 2,6-di-F-4-OMe—Ph, R² is Me, R³ and R⁴ are both F. |
| 327 | Q¹ is 2,6-di-F-4-OMe—Ph, R² is Cl, R³ and R⁴ are both Cl. |
| 328 | Q¹ is 2,6-di-F-4-OMe—Ph, R² is Br, R³ and R⁴ are both Br. |
| 329 | Q¹ is 2,6-di-F-4-OEt—Ph, R² is Me, R³ is F, and R⁴ is Cl. |
| 330 | Q¹ is 2,6-di-F-4-OEt—Ph, R² is Br, R³ and R⁴ are both F. |
| 331 | Q¹ is 2,6-di-F-4-CN—Ph, R² is Me, R³ and R⁴ are both Cl. |
| 332 | Q¹ is 2,6-di-F-4-CN—Ph, R² is Cl, R³ and R⁴ are both Br. |
| 333 | Q¹ is 2,6-di-F-4-CN—Ph, R² is Br, R³ is F, and R⁴ is Cl. |
| 334 | Q¹ is 2-Cl-4,6-di-F—Ph, R² is Cl, R³ and R⁴ are both F. |
| 335 | Q¹ is 2-Cl-4,6-di-F—Ph, R² is Br, R³ and R⁴ are both Cl. |
| 336 | Q¹ is 2-Cl-2,6-di-F—Ph, R² is Me, R³ and R⁴ are both Br. |
| 337 | Q¹ is 2-Cl-2,6-di-F—Ph, R² is Cl, R³ is F, and R⁴ is Cl. |
| 338 | Q¹ is 2-F-4-MeO—Ph, R² is Me, R³ and R⁴ are both F. |
| 339 | Q¹ is 2-F-4-MeO—Ph, R² is Cl, R³ and R⁴ are both Cl. |
| 340 | Q¹ is 2-F-4-MeO—Ph, R² is Br, R³ and R⁴ are both Br. |
| 341 | Q¹ is 2-F-4-EtO—Ph, R² is Me, R³ is F, and R⁴ is Cl. |
| 342 | Q¹ is 2-F-4-EtO—Ph, R² is Br, R³ and R⁴ are both F. |
| 343 | Q¹ is 2-Cl-4-MeO—Ph, R² is Me, R³ and R⁴ are both Cl. |
| 344 | Q¹ is 2-Cl-4-MeO—Ph, R² is Cl, R³ and R⁴ are both Br. |
| 345 | Q¹ is 2-Cl-4-MeO—Ph, R² is Br, R³ is F, and R⁴ is Cl. |
| 346 | Q¹ is 2-Cl-4-EtO—Ph, R² is Cl, R³ and R⁴ are both F. |
| 347 | Q¹ is 2-Cl-4-EtO—Ph, R² is Br, R³ and R⁴ are both Cl. |
| 348 | Q¹ is 2-Br-4-MeO—Ph, R² is Me, R³ and R⁴ are both Br. |
| 349 | Q¹ is 2-Br-4-MeO—Ph, R² is Cl, R³ is F, and R⁴ is Cl. |
| 350 | Q¹ is 2-Br-4-EtO—Ph, R² is Me, R³ and R⁴ are both F. |
| 351 | Q¹ is 2-Br-4-EtO—Ph, R² is Cl, R³ and R⁴ are both Cl. |
| 352 | Q¹ is 2-Br-4-EtO—Ph, R² is Br, R³ and R⁴ are both Br. |
| 353 | Q¹ is 2-F-4-CN—Ph, R² is Me, R³ is F, and R⁴ is Cl. |
| 354 | Q¹ is 2-F-4-CN—Ph, R² is Br, R³ and R⁴ are both F. |
| 355 | Q¹ is 2-Cl-4-CN—Ph, R² is Me, R³ and R⁴ are both Cl. |
| 356 | Q¹ is 2-Cl-4-CN—Ph, R² is Cl, R³ and R⁴ are both Br. |
| 357 | Q¹ is 2-Cl-4-CN—Ph, R² is Br, R³ is F, and R⁴ is Cl. |
| 358 | Q¹ is 2-Br-4-CN—Ph, R² is Cl, R³ and R⁴ are both F. |
| 359 | Q¹ is 2-Br-4-CN—Ph, R² is Br, R³ and R⁴ are both Cl. |
| 360 | Q¹ is 2,5-di-Cl-3-Py, R² is Me, R³ and R⁴ are both Br. |
| 361 | Q¹ is 2,5-di-Cl-3-Py, R² is Cl, R³ is F, and R⁴ is Cl. |
| 362 | Q¹ is 2-Cl-3-Th, R² is Me, R³ and R⁴ are both F. |
| 363 | Q¹ is 2-Cl-3-Th, R² is Cl, R³ and R⁴ are both Cl. |
| 364 | Q¹ is 2-Cl-3-Th, R² is Br, R³ and R⁴ are both Br. |
| 365 | Q¹ is 2,5-di-Cl-3-Th, R² is Cl, R³ and R⁴ are both F. |
| 366 | Q¹ is 2,5-di-Cl-3-Th, R² is Br, R³ and R⁴ are both Cl. |
| 367 | Q¹ is 2,4-di-F—Ph, R² is Me, R³ is Me, and R⁴ is F. |
| 368 | Q¹ is 2,4-di-F—Ph, R² is Br, R³ is Me and R⁴ is Cl. |
| 369 | Q¹ is 2,4,6-tri-F—Ph, R² is Me, R³ is Me and R⁴ is I. |
| 370 | Q¹ is 2,4,6-tri-F—Ph, R² is Cl, R³ is Et and R⁴ is F. |
| 371 | Q¹ is 2,4,6-tri-F—Ph, R² is Me, R³ is Et, and R⁴ is Cl. |
| 372 | Q¹ is 2-F-4-Cl—Ph, R² is Me, R³ is Et, and R⁴ is I. |
| 373 | Q¹ is 2-F-4-Cl—Ph, R² is Me, R³ is n-Pr and R⁴ is F. |
| 374 | Q¹ is 2-Br-4-F—Ph, R² is Me, R³ is n-Pr and R⁴ is Cl. |
| 375 | Q¹ is 2-Br-4-F—Ph, R² is Me, R³ is i-Pr and R⁴ is F. |
| 376 | Q¹ is 2-Br-4-Cl—Ph, R² is Me, R³ is i-Pr, and R⁴ is Cl. |
| 377 | Q¹ is 2-Br-6-F—Ph, R² is Me, R³ is n-Bu and R⁴ is F. |
| 378 | Q¹ is 2-Cl-4-F—Ph, R² is Me, R³ is n-Bu and R⁴ is Cl. |

TABLE 379

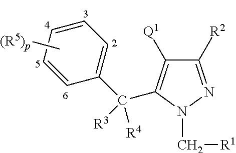

Q¹ is 2,6-di-F—Ph, R¹ is Me, R² is Me, R³ is H, and R⁴ is F.

| (R⁵)ₚ | (R⁵)ₚ |
|---|---|
| 2-F | 2-F-4-CN |
| 4-Cl | 2-Cl-4,6-di-F |
| 2,6-di-F | 4-Br-2,5-di-F |
| 2-Cl-4-F | 2-CF₃-4-F |
| 2-Br-4-F | 2-Br-4-Cl |
| 2-Br-4-MeO | 2-Cl |
| 2,6-di-F-4-CN | 4-Br |
| 4-Cl-2,5-di-F | 2,3,5-tri-F |
| 2,6-di-Cl-4-F | 2,6-di-Cl |
| 2-F-4-Br | 2-F-4-MeO |
| 3-F | 2-Cl-4-CN |
| 2-Br | 2-Br-4,5-di-F |
| 2,4,6-tri-F | 4-Br-2,6-di-F |
| 2-F-4-Cl | 4-Me |
| 2-I-4-F | 2-Br-4-F-6-Cl |
| 2,6-di-F-4-MeO | 3-Cl |
| 2-Cl-4,5-di-F | 2,4-di-F |
| 4-Cl-2,6-di-F | 2,3,6-tri-F |
| 2,6-di-Cl-4-MeO | 2,4,6-tri-Cl |
| 2-Cl-4-Br | 2-Cl-4-MeO |
| 4-F | 2-Br-4-CN |
| 3-Br | 2-Br-4,6-di-F |
| 2,4,5-tri-F | 2,4-di-Cl-6-F |
| 2,4-di-Cl | 2,4-di-Me |
| 2-Me-4-F | 2-Cl-4-Br-6-F |

The present disclosure also includes Tables 380 through 416, each of which is constructed the same as Table 379 above, except that the row heading in Table 379 (i.e. “Q¹ is 2,6-di-F-Ph, R¹ is Me, R² is Me, R³ is H, and R⁴ is F.”) is replaced with the respective row heading shown below. For example, in Table 380 the row heading is “Q¹ is 2,6-di-F-Ph, R¹ is Me, R² is Me, R³ is H, and R⁴ is Br”, and (R⁵)ₚ is as defined in Table 379 above. Thus, the first entry in Table 380 specifically discloses 5-[chloro(2-fluorophenyl)methyl]-4-(2,6-difluorophenyl)-1-ethyl-3-methyl-1H-pyrazole. Tables 381 through 416 are constructed similarly.

| Table | Row Heading |
|---|---|
| 380 | Q¹ is 2,6-di-F—Ph, R¹ is Me, R² is Me, R³ is H, and R⁴ is Cl. |
| 381 | Q¹ is 2,6-di-F—Ph, R¹ is Me, R² is Me, R³ is H, and R⁴ is Br. |
| 382 | Q¹ is 2,6-di-F—Ph, R¹ is Me, R² is Me, R³ is H, and R⁴ is I. |
| 383 | Q¹ is 2,6-di-F—Ph, R¹ is Me, R² is Me, R³ and R⁴ are both F. |
| 384 | Q¹ is 2,6-di-F—Ph, R¹ is Me, R² is Me, R³ is F, and R⁴ is Cl. |
| 385 | Q¹ is 2,6-di-F—Ph, R¹ is Me, R² is Me, R³ is Me, and R⁴ is Cl. |
| 386 | Q¹ is 2,4-di-F—Ph, R¹ is Me, R² is Br, R³ is H, and R⁴ is F. |
| 387 | Q¹ is 2,4-di-F—Ph, R¹ is Me, R² is Br, R³ and R⁴ are both Cl. |
| 388 | Q¹ is 2,4-di-F—Ph, R¹ is Me, R² is Cl, R³ is H, and R⁴ is F. |
| 389 | Q¹ is 2,4,6-tri-F—Ph, R¹ is Me, R² is Cl, R³ is H, and R⁴ is F. |
| 390 | Q¹ is 2-Cl—Ph, R¹ is Me, R² is Cl, R³ is H, and R⁴ is F. |
| 391 | Q¹ is 2-Br—Ph, R¹ is Me, R² is Cl, R³ is Me, and R⁴ is F. |
| 392 | Q¹ is 2-F-4-Cl—Ph, R¹ is Me, R² is Cl, R³ is Me, and R⁴ is Cl. |
| 393 | Q¹ is 2-Br-4-Cl—Ph, R¹ is Me, R² is Cl, R³ and R⁴ are both I. |
| 394 | Q¹ is 2-Br-6-F—Ph, R¹ is Me, R² is Cl, R³ is H, and R⁴ is Cl. |
| 395 | Q¹ is 2-Cl-4-F—Ph, R¹ is Me, R² is Cl, R³ is Me, and R⁴ is F. |
| 396 | Q¹ is 2-Cl-6-F—Ph, R¹ is Me, R² is Cl, R³ and R⁴ are both F. |
| 397 | Q¹ is 2,4-di-Cl—Ph, R¹ is Me, R² is Cl, R³ is Me, and R⁴ is F. |
| 398 | Q¹ is 2-I-4-F—Ph, R¹ is Me, R² is Cl, R³ is H, and R⁴ is F. |
| 399 | Q¹ is 2-Me-4-F—Ph, R¹ is Me, R² is Cl, R³ is H, and R⁴ is F. |
| 400 | Q¹ is 2,6-di-Cl—Ph, R¹ is Me, R² is Cl, R³ is Me, and R⁴ is Cl. |
| 401 | Q¹ is 2,6-di-F-4-MeO—Ph, R¹ is Me, R² is Cl, R³ and R⁴ are |
| | both Br. |
| 402 | Q¹ is 2,6-di-F-4-EtO—Ph, R¹ is Me, R² is Cl, R³ is H, and R⁴ is F. |
| 403 | Q¹ is 2,6-di-F-4-CN—Ph, R¹ is Me, R² is Cl, R³ is H, and R⁴ is F. |
| 404 | Q¹ is 2-Cl-4,6-di-F—Ph, R¹ is Me, R² is Cl, R³ is H, and R⁴ is F. |
| 405 | Q¹ is 2-F-4-MeO—Ph, R¹ is Me, R² is Cl, R³ is Me, and R⁴ is Cl. |
| 406 | Q¹ is 2-F-4-EtO—Ph, R¹ is Me, R² is Cl, R³ is H, and R⁴ is Cl. |
| 407 | Q¹ is 2,5-di-Cl-3-Py, R¹ is Me, R² is Cl, R³ is H, and R⁴ is F. |
| 408 | Q¹ is 2,5-di-Cl-3-Th, R¹ is Me, R² is Cl, R³ is F, and R⁴ is F. |
| 409 | Q¹ is 2-Cl-4-F—Ph, R¹ is H, R² is F, R³ is H, and R⁴ is Cl. |
| 410 | Q¹ is 2-Cl-6-F—Ph, R¹ is H, R² is F, R³ is H, and R⁴ is F. |
| 411 | Q¹ is 2-Cl-4-F—Ph, R¹ is H, R² is I, R³ is H, and R⁴ is Cl. |
| 412 | Q¹ is 2-Cl-6-F—Ph, R¹ is H, R² is I, R³ is H, and R⁴ is F. |
| 413 | Q¹ is 2-Cl-4-F—Ph, R¹ is H, R² is Me, R³ is Br, and R⁴ is Cl. |
| 414 | Q¹ is 2-Cl-6-F—Ph, R¹ is H, R² is Me, R³ is Cl, and R⁴ is I. |
| 415 | Q¹ is 2-Cl-4-F—Ph, R¹ is H, R² is Me, R³ is F, and R⁴ is Cl. |
| 416 | Q¹ is 2-Cl-6-F—Ph, R¹ is H, R² is Me, R³ is Br, and R⁴ is I. |

### Formulation/Utility

A compound of this invention will generally be used as a fungicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g, oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000. In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 2 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 2 | 10.0% |
| attapulgite granules (low volatile matter, | 90.0% |
| 0.71/0.30 mm; U.S.S. No. 25-50 sieves) | |

### Example D

Extruded Pellet

| | |
|---|---|
| Compound 3 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 1 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 4 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 5 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Formulations such as those in the Formulation Table are typically diluted with water to form aqueous compositions before application. Aqueous compositions for direct applications to the plant or portion thereof (e.g., spray tank compositions) typically at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rutstroemia floccosum (also known as Sclerontina homoeocarpa); Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Accordingly, this aspect of the present invention can also be described as a method for protecting a plant or plant seed from diseases caused by fungal pathogens comprising applying a fungicidally effective amount of a compound of Formula 1, an N-oxide, or salt thereof to the plant (or portion thereof) or plant seed (directly or through the environment (e.g., growing medium) of the plant or plant seed).

Rates of application for these compounds can be influenced by many factors of the environment and should be determined under actual use conditions. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a fungicidally effective amount of a compound of Formula 1 and a biologically effective amount of at least one additional biologically active compound or agent and can further comprise at least one of a surfactant, a solid diluent or a liquid diluent.

The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

As mentioned in the Summary of the Invention, one aspect of the present invention is a fungicidal composition comprising (i.e. a mixture or combination of) a compound of Formula 1, an N-oxide, or a salt thereof (i.e. component a), and at least one other fungicide (i.e. component b).

Of note is a composition which, in addition to the Formula 1 compound of component (a), includes as component (b) at least one fungicidal compound selected from the group consisting of the classes (b1) methyl benzimidazole carbamate (MBC) fungicides; (b2) dicarboximide fungicides; (b3) demethylation inhibitor (DMI) fungicides; (b4) phenylamide fungicides; (b5) amine/morpholine fungicides; (b6) phospholipid biosynthesis inhibitor fungicides; (b7) carboxamide fungicides; (b8) hydroxy(2-amino-)pyrimidine fungicides; (b9) anilinopyrimidine fungicides; (b10) N-phenyl carbamate fungicides; (b11) quinone outside inhibitor (QoI) fungicides; (b12) phenylpyrrole fungicides; (b13) quinoline fungicides; (b14) lipid peroxidation inhibitor fungicides; (b15) melanin biosynthesis inhibitors-reductase (MBI-R) fungicides; (b16) melanin biosynthesis inhibitors-dehydratase (MBI-D) fungicides; (b 17) hydroxyanilide fungicides; (b 18) squalene-epoxidase inhibitor fungicides; (b 19) polyoxin fungicides; (b20) phenylurea fungicides; (b21) quinone inside inhibitor (QiI) fungicides; (b22) benzamide fungicides; (b23) enopyranuronic acid antibiotic fungicides; (b24) hexopyranosyl antibiotic fungicides; (b25) glucopyranosyl antibiotic: protein synthesis fungicides; (b26) glucopyranosyl antibiotic: trehalase and inositol biosynthesis fungicides; (b27) cyanoacetamideoxime fungicides; (b28) carbamate fungicides; (b29) oxidative phosphorylation uncoupling fungicides; (b30) organo tin fungicides; (b31) carboxylic acid fungicides; (b32) heteroaromatic fungicides; (b33) phosphonate fungicides; (b34) phthalamic acid fungicides; (b35) benzotriazine fungicides; (b36) benzene-sulfonamide fungicides; (b37) pyridazinone fungicides; (b38) thiophene-carboxamide fungicides; (b39) pyrimidinamide fungicides; (b40) carboxylic acid amide (CAA) fungicides; (b41) tetracycline antibiotic fungicides; (b42) thiocarbamate fungicides; (b43) benzamide fungicides; (b44) host plant defense induction fungicides; (b45) multi-site contact activity fungicides; (b46) fungicides other than classes (b1) through (b45); and salts of compounds of classes (b1) through (b46).

Further descriptions of these classes of fungicidal compounds are provided below.

(b1) “Methyl benzimidazole carbamate (MBC) fungicides” (FRAC (Fungicide Resistance Action Committee) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(b2) “Dicarboximide fungicides” (FRAC code 2) are proposed to inhibit a lipid peroxidation in fungi through interference with NADH cytochrome c reductase. Examples include chlozolinate, iprodione, procymidone and vinclozolin.

(b3) “Demethylation inhibitor (DMI) fungicides” (FRAC code 3) inhibit C14-demethylase which plays a role in sterol production. Sterols, such as ergosterol, are needed for membrane structure and function, making them essential for the development of functional cell walls. Therefore, exposure to these fungicides result in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz, pefurazoate and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(b4) “Phenylamide fungicides” (FRAC code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl, metalaxyl-M (also known as mefenoxam). The oxazolidinones include oxadixyl. The butyrolactones include ofurace.

(b5) “Amine/morpholine fungicides” (FRAC code 5) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(b6) “Phospholipid biosynthesis inhibitor fungicides” (FRAC code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(b7) “Carboxamide fungicides” (FRAC code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamide, furan carboxamide, oxathiin carboxamide, thiazole carboxamide, pyrazole carboxamide and pyridine carboxamide. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include bixafen, furametpyr, isopyrazam, fluxapyroxad, sedaxane (N-[2-(1S,2R)-[1,1′-bicyclopropyl]-2-ylphenyl]-3-(difluoromethyl)-1-methyl-1H-pyrazole-4-carboxamide) and penflufen (N-[2-(1,3-dimethylbutyl)phenyl]-5-fluoro-1,3-dimethyl-1H-pyrazole-4-carboxamide (PCT Patent Publication WO 2003/010149)). The pyridine carboxamides include boscalid.

(b8) “Hydroxy(2-amino-)pyrimidine fungicides” (FRAC code 8) inhibit nucleic acid synthesis by interfering with adenosine deaminase. Examples include bupirimate, dimethirimol and ethirimol.

(b9) “Anilinopyrimidine fungicides” (FRAC code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(b10) “N-Phenyl carbamate fungicides” (FRAC code 10) inhibit mitosis by binding to 13-tubulin and disrupting microtubule assembly. Inhibition of microtubule assembly can disrupt cell division, transport within the cell and cell structure. Examples include diethofencarb.

(b 11) “Quinone outside inhibitor (QoI) fungicides” (FRAC code 11) inhibit Complex III mitochondrial respiration in fungi by affecting ubiquinol oxidase. Oxidation of ubiquinol is blocked at the “quinone outside” (Qₒ) site of the cytochrome bc₁ complex, which is located in the inner mitochondrial membrane of fungi Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071) and picoxystrobin. The methoxycarbamates include pyraclostrobin and pyrametostrobin. The oximinoacetates include kresoxim-methyl, pyraoxystrobin and trifloxystrobin. The oximinoacetamides include dimoxystrobin, metominostrobin, orysastrobin, a-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(b12) “Phenylpyrrole fungicides” (FRAC code 12) inhibit a MAP protein kinase associated with osmotic signal transduction in fungi. Fenpiclonil and fludioxonil are examples of this fungicide class. (b13) “Quinoline fungicides” (FRAC code 13) are proposed to inhibit signal transduction by affecting G-proteins in early cell signaling. They have been shown to interfere with germination and/or appressorium formation in fungi that cause powder mildew diseases. Quinoxyfen is an example of this class of fungicide.

(b14) “Lipid peroxidation inhibitor fungicides” (FRAC code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(b15) “Melanin biosynthesis inhibitors-reductase (MBI-R) fungicides” (FRAC code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(b16) “Melanin biosynthesis inhibitors-dehydratase (MBI-D) fungicides” (FRAC code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(b17) “Hydroxyanilide fungicides (FRAC code 17) inhibit C4-demethylase which plays a role in sterol production. Examples include fenhexamid.

(b18) “Squalene-epoxidase inhibitor fungicides” (FRAC code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(b19) “Polyoxin fungicides” (FRAC code 19) inhibit chitin synthase. Examples include polyoxin.

(b20) “Phenylurea fungicides” (FRAC code 20) are proposed to affect cell division. Examples include pencycuron.

(b21) “Quinone inside inhibitor (QiI) fungicides” (FRAC code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(b22) “Benzamide fungicides” (FRAC code 22) inhibit mitosis by binding to 13-tubulin and disrupting microtubule assembly Inhibition of microtubule assembly can disrupt cell division, transport within the cell and cell structure. Examples include zoxamide.

(b23) “Enopyranuronic acid antibiotic fungicides” (FRAC code 23) inhibit growth of fungi by affecting protein biosynthesis. Examples include blasticidin-S.

(b24) “Hexopyranosyl antibiotic fungicides” (FRAC code 24) inhibit growth of fungi by affecting protein biosynthesis. Examples include kasugamycin.

(b25) “Glucopyranosyl antibiotic: protein synthesis fungicides” (FRAC code 25) inhibit growth of fungi by affecting protein biosynthesis. Examples include streptomycin.

(b26) “Glucopyranosyl antibiotic: trehalase and inositol biosynthesis fungicides” (FRAC code 26) inhibit trehalase in inositol biosynthesis pathway. Examples include validamycin.

(b27) “Cyanoacetamideoxime fungicides (FRAC code 27) include cymoxanil.

(b28) “Carbamate fungicides” (FRAC code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(b29) “Oxidative phosphorylation uncoupling fungicides” (FRAC code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(b30) “Organo tin fungicides” (FRAC code 30) inhibit adenosine triphosphate (ATP) synthase in oxidative phosphorylation pathway. Examples include fentin acetate, fentin chloride and fentin hydroxide.

(b31) “Carboxylic acid fungicides” (FRAC code 31) inhibit growth of fungi by affecting deoxyribonucleic acid (DNA) topoisomerase type II (gyrase). Examples include oxolinic acid.

(b32) “Heteroaromatic fungicides” (FRAC code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(b33) “Phosphonate fungicides” (FRAC code 33) include phosphorous acid and its various salts, including fosetyl-aluminum.

(b34) “Phthalamic acid fungicides” (FRAC code 34) include teclofthalam.

(b35) “Benzotriazine fungicides” (FRAC code 35) include triazoxide.

(b36) “Benzene-sulfonamide fungicides” (FRAC code 36) include flusulfamide.

(b37) “Pyridazinone fungicides” (Fungicide Resistance Action Committee (FRAC) code 37) include diclomezine.

(b38) “Thiophene-carboxamide fungicides” (FRAC code 38) are proposed to affect ATP production. Examples include silthiofam.

(b39) “Pyrimidinamide fungicides” (FRAC code 39) inhibit growth of fungi by affecting phospholipid biosynthesis and include diflumetorim.

(b40) “Carboxylic acid amide (CAA) fungicides” (FRAC code 40) are proposed to inhibit phospholipid biosynthesis and cell wall deposition Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb and valifenalate (valiphenal). The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)-amino]butanamide and N-[2-[4-[[3-(4-chlorophenyl)-2-propyn-1-yl]oxy]-3-methoxyphenyl]-ethyl]-3-methyl-2-[(ethylsulfonyl)amino]butanamide.

(b41) “Tetracycline antibiotic fungicides” (FRAC code 41) inhibit growth of fungi by affecting complex 1 nicotinamide adenine dinucleotide (NADH) oxidoreductase. Examples include oxytetracycline.

(b42) “Thiocarbamate fungicides (b42)” (FRAC code 42) include methasulfocarb.

(b43) “Benzamide fungicides” (FRAC code 43) inhibit growth of fungi by delocalization of spectrin-like proteins. Examples include acylpicolide fungicides such as fluopicolide and fluopyram.

(b44) “Host plant defense induction fungicides” (FRAC code P) induce host plant defense mechanisms. Host plant defense induction fungicides include benzo-thiadiazole, benzisothiazole and thiadiazole-carboxamide fungicides. The benzo-thiadiazoles include acibenzolar-S-methyl. The benzisothiazoles include probenazole. The thiadiazole-carboxamides include tiadinil and isotianil.

(b45) “Multi-site contact fungicides” inhibit fungal growth through multiple sites of action and have contact/preventive activity. This class of fungicides includes: (b45.1) “copper fungicides” (FRAC code M1)”, (b45.2) “sulfur fungicides” (FRAC code M2), (b45.3) “dithiocarbamate fungicides” (FRAC code M3), (b45.4) “phthalimide fungicides” (FRAC code M4), (b45.5) “chloronitrile fungicides” (FRAC code M5), (b45.6) “sulfamide fungicides” (FRAC code M6), (b45.7) “guanidine fungicides” (FRAC code M7), (b45.8) “triazine fungicides” (FRAC code M8) and (b45.9) “quinone fungicides” (FRAC code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine and imoctadine, including iminoctadine albesilate and iminoctadine triacetate. “Triazine fungicides” include anilazine. “Quinone fungicides” include dithianon.

(b46) “Fungicides other than fungicides of classes (b1) through (b45)” include certain fungicides whose mode of action may be unknown. These include: (b46.1) “thiazole carboxamide fungicides” (FRAC code U5), (b46.2) “phenyl-acetamide fungicides” (FRAC code U6), (b46.3) “quinazolinone fungicides” (FRAC code U7) and (b46.4) “benzophenone fungicides” (FRAC code U8). The thiazole carboxamides include ethaboxam. The phenyl-acetamides include cyflufenamid and N-[[(cyclopropylmethoxy)amino][6-(difluoromethoxy)-2,3-difluorophenyl]-methylene]benzeneacetamide. The quinazolinones include proquinazid and 2-butoxy-6-iodo-3-propyl-4H-1-b enzopyran-4-one. The benzophenones include metrafenone and pyriofenone. The (b46) class also includes bethoxazin, neo-asozin (ferric methanearsonate), fenpyrazamine, pyrrolnitrin, quinomethionate, tebufloquin, N-[2-[4-[[3-(4-chlorophenyl)-2-propyn-1-yl]oxy]-3-methoxy-phenyl]ethyl]-3-methyl-2-[(methylsulfonyl)amino]butanamide, N-[2-[4-[[3-(4-chloro-phenyl)-2-propyn-1-yl]oxy]-3-methoxyphenyl]ethyl]-3-methyl-2-[(ethylsulfonyl)amino]-butanamide, 2-[[2-fluoro-5-(trifluoromethyl)phenyl]thio]-2-[3-(2-methoxyphenyl)-2-thiazo-lidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl) [1,2,4]triazolo[1,5-c]pyrimidine, N-(4-chloro-2-nitrophenyl)-N-ethyl-4-methylbenzenesulfonamide, N-[[(cyclopropylmethoxy)-amino][6-(difluoromethoxy)-2,3-difluorophenyl]methylene]benzeneacetamide, N-[4-[4-chloro-3-(trifluoromethyl)phenoxy]-2,5-dimethylphenyl]-N-ethyl-N-methylmethanimid-amide and 1-[(2-propenylthio)carbonyl]-2-(1-methylethyl)-4-(2-methylphenyl)-5-amino-1H-pyrazol-3-one.

Therefore of note is a mixture (i.e. composition) comprising as component (a) a compound of Formula 1 (or an N-oxide or salt thereof) and as component (b) at least one fungicidal compound selected from the group consisting of the aforedescribed classes (b1) through (b46). Also of note are embodiments wherein component (b) comprises at least one fungicide from each of two different groups selected from (b1) through (b46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (b1) through (b46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, acetoprole, aldicarb, amidoflumet (S-1955), amitraz, avermectin, azadirachtin, azinphos-methyl, bifenthrin, bifenazate, bistrifluoron, buprofezin, carbofuran, cartap, chinomethionat, chlorfenapyr, chlorfluazuron, chlorantraniliprole (DPX-E2Y45), chlorpyrifos, chlorpyrifos-methyl, chlorobenzilate, chromafenozide, clothianidin, cyantraniliprole (3-bromo-1-(3-chloro-2-pyridinyl)-N-[4-cyano-2-methyl-6-[(methylamino)-carbonyl]phenyl]-1H-pyrazole-5-carboxamide), cyflumetofen, cyfluthrin, beta-cyfluthrin, cyhalothrin, gamma-cyhalothrin, lambda-cyhalothrin, cyhexatin, cypermethrin, cyromazine, deltamethrin, diafenthiuron, diazinon, dicofol, dieldrin, dienochlor, diflubenzuron, dimefluthrin, dimethoate, dinotefuran, diofenolan, emamectin, endosulfan, esfenvalerate, ethiprole, etoxazole, fenamiphos, fenazaquin, fenbutatin oxide, fenothiocarb, fenoxycarb, fenpropathrin, fenpyroximate, fenvalerate, fipronil, flonicamid, flubendiamide, flucythrinate, tau-fluvalinate, flufenerim (UR-50701), flufenoxuron, fonophos, halofenozide, hexaflumuron, hexythiazox, hydramethylnon, imicyafos, imidacloprid, indoxacarb, isofenphos, lufenuron, malathion, metaflumizone, metaldehyde, methamidophos, methidathion, methomyl, methoprene, methoxychlor, methoxyfenozide, metofluthrin, monocrotophos, nitenpyram, nithiazine, novaluron (XDE-007), noviflumuron, oxamyl, parathion, parathion-methyl, permethrin, phorate, phosalone, phosmet, phosphamidon, pirimicarb, profenofos, profluthrin, propargite, prothiocarb, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spiridiclofen, spiromesifen (BSN 2060), spirotetramat, sulprofos, tebufenozide, tebufenpyrad, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon, triflumuron; nematocides such as aldicarb, imicyafos, oxamyl and fenamiphos; bactericides such as streptomycin; acaricides such as amitraz, chinomethionat, chlorobenzilate, cyenopyrafen, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 (or an N-oxide or salt thereof) is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

Of note is a combination of a compound of Formula 1 (or an N-oxide or salt thereof) with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to a compound of Formula 1 include at least one compound selected from the group consisting of (1) alkylenebis(dithiocarbamate) fungicides; (2) cymoxanil; (3) phenylamide fungicides; (4) pyrimidinone fungicides; (5) chlorothalonil; (6) carboxamides acting at complex II of the fungal mitochondrial respiratory electron transfer site; (7) quinoxyfen; (8) metrafenone or pyriofenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Pyrimidinone fungicides (group (4)) include compounds of Formula A1

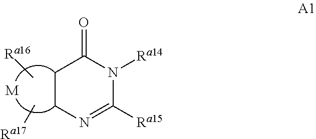

wherein M forms a fused phenyl, thiophene or pyridine ring; Ra¹⁴ is C₁-C₆ alkyl; Ra¹⁵ is C₁-C₆ alkyl or C₁-C₆ alkoxy; Ra¹⁶ is halogen; and Ra¹⁷ is hydrogen or halogen.

Pyrimidinone fungicides are described in PCT Patent Application Publication WO 94/26722 and U.S. Pat. Nos. 6,066,638, 6,245,770, 6,262,058 and 6,277,858. Of note are pyrimidinone fungicides selected from the group: 6-bromo-3-propyl-2-propyloxy-4(3H)-quinazolinone, 6,8-diiodo-3-propyl-2-propyloxy-4(3H)-quinazolinone, 6-iodo-3-propyl-2-propyloxy-4(3H)-quinazolinone (proquinazid), 6-chloro-2-propoxy-3-propyl-thieno[2,3-c/]pyrimidin-4(3H)-one, 6-bromo-2-propoxy-3-propylthieno[2,3-c/]pyrimidin-4(3H)-one, 7-bromo-2-propoxy-3-propylthieno[3,2-c/]pyrimidin-4(3H)-one, 6-bromo-2-propoxy-3-propylpyrido[2,3-d]pyrimidin-4(3H)-one, 6,7-dibromo-2-propoxy-3-propyl-thieno[3,2-c/]pyrimidin-4(3H)-one, and 3-(cyclopropylmethyl)-6-iodo-2-(propylthio)pyrido-[2,3-d]pyrimidin-4(3H)-one.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, fluxapyroxad, furametpyr, mepronil, oxycarboxin, thifluzamide, penthiopyrad and penflufen (N-[2-(1,3-dimethylbutyl)phenyl]-5-fluoro-1,3-dimethyl-1H-pyrazole-4-carboxamide (PCT Patent Publication WO 2003/010149)), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin.

Examples of component (b) fungicides include acibenzolar-5-methyl, aldimorph, ametoctradin, amisulbrom, anilazine, azaconazole, azoxystrobin, benalaxyl, benalaxyl-M, benodanil, benomyl, benthiavalicarb, benthiavalicarb-isopropyl, bethoxazin, binapacryl, biphenyl, bitertanol, bixafen, blasticidin-S, boscalid, bromuconazole, bupirimate, carboxin, carpropamid, captafol, captan, carbendazim, chloroneb, chlorothalonil, chlozolinate, clotrimazole, copper salts such as Bordeaux mixture (tribasic copper sulfate), copper hydroxide and copper oxychloride, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole, diniconazole-M, dinocap, dithianon, dodemorph, dodine, edifenphos, enestroburin, epoxiconazole, ethaboxam, ethirimol, etridiazole, famoxadone, fenamidone, fenarimol, fenbuconazole, fenfuram, fenhexamid, fenoxanil, fenpiclonil, fenpropidin, fenpropimorph, fenpyrazamine, fentin acetate, fentin chloride, fentin hydroxide, ferbam, ferimzone, fluazinam, fludioxonil, flumetover, flumorph, fluopicolide (also known as picobenzamid), fluopyram, fluoroimide, fluoxastrobin, fluquinconazole, flusilazole, flusulfamide, flutianil (2-[[2-fluoro-5-(trifluoromethyl)phenyl]thio]-2-[3-(2-methoxyphenyl)-2-thiazolidinylidene]acetonitrile), flutolanil, flutriafol, fluxapyroxad, folpet, fosetyl-aluminum, fuberidazole, furalaxyl, furametpyr, hexaconazole, hymexazol, guazatine, imazalil, imibenconazole, iminoctadine, iodocarb, ipconazole, iprobenfos, iprodione, iprovalicarb, isoprothiolane, isopyrazam, isotianil, kasugamycin, kresoxim-methyl, mancozeb, mandipropamid, maneb, mepronil, meptyldinocap, metalaxyl, metalaxyl-M, metconazole, methasulfocarb, metiram, metominostrobin, mepanipyrim, metrafenone, myclobutanil, naftifine, neo-asozin (ferric methanearsonate), nuarimol, octhilinone, ofurace, orysastrobin, oxadixyl, oxolinic acid, oxpoconazole, oxycarboxin, oxytetracycline, penconazole, pencycuron, penflufen, penthiopyrad, pefurazoate, phosphorous acid and salts, phthalide, picoxystrobin, piperalin, polyoxin, probenazole, prochloraz, procymidone, propamocarb, propamocarb-hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyriofenone, pyroquilon, pyrrolnitrin, quinomethionate, quinoxyfen, quintozene, sedaxane, silthiofam, simeconazole, spiroxamine, streptomycin, sulfur, tebuconazole, tebufloquin, tecloftalam, tecnazene, terbinafine, tetraconazole, thiabendazole, thifluzamide, thiophanate, thiophanate-methyl, thiram, tiadinil, tolclofos-methyl, tolylfluanid, triadimefon, triadimenol, triazoxide, tricyclazole, tridemorph, triflumizole, tricyclazole, trifloxystrobin, triforine, trimorphamide, triticonazole, uniconazole, validamycin, valifenalate (valiphenal), vinclozolin, zineb, ziram, zoxamide, N-[4-[4-chloro-3-(trifluoromethyl)phenoxy]-2,5-dimethylphenyl]-N-ethyl-N-methylmethanimidamide, 5-chloro-6-(2,4,6-trifluorophenyl)-7-(4-methylpiperidin-1-yl)[1,2,4]triazolo[1,5-c]pyrimidine (BAS 600), penflufen (N-[2-(1,3-dimethylbutyl)phenyl]-5-fluoro-1,3-dimethyl-1H-pyrazole-4-carboxamide), N-[2-[4-[[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino ]butanamide, 2-butoxy-6-iodo-3-propyl-4H-1-benzopyran-4-one, 3-[5-(4-chlorophenyl)-2,3-dimethyl-3-isoxazolidinyl]-pyridine, 4-fluorophenyl N-[1-[[[1-(4-cyanophenyl)ethyl]sulfonyl]methyl]propyl]carbamate, N-[[(cyclopropylmethoxy)amino][6-(difluoromethoxy)-2,3-difluoro-phenyl]methylene]benzeneacetamide, α-(methoxyimino)-N-methyl-2-[[[1-[3-(trifluoro-methyl)phenyl]ethoxy]imino]methyl]benzeneacetamide, N-[4-[4-chloro-3-(trifluoro-methyl)phenoxy]-2,5-dimethylphenyl]-N-ethyl-N-methylmethanimidamide, N-(4-chloro-2-nitrophenyl)-N-ethyl-4-methylbenzenesulfonamide, 2-[[[[3-(2,6-dichlorophenyl)-1-methyl-2-propen-1-ylidene]amino]oxy]methyl]-α-(methoxyimino)-N-methylbenzeneacetamide, 1-[(2-propenylthio)carbonyl]-2-(1-methylethyl)-4-(2-methylphenyl)-5-amino-1H-pyrazol-3-one, ethyl-6-octyl-[1,2,4]triazolo[1,5-a]pyrimidin-7-ylamine, pentyl N-[4-[[[[(1-methyl-1H-tetrazol-5-yl)phenylmethylene]amino]oxy]methyl]-2-thiazolyl]carbamate and pentyl N-[6-[[[[(1-methyl-1H-tetrazol-5-yl)phenylmethylene]amino]oxy]methyl]-2-pyridinyl]carbamate

Of note are combinations of compounds of Formula 1 (or an N-oxide or salt thereof) (i.e. Component (a) in compositions) with azoxystrobin, kresoxim-methyl, trifloxystrobin, pyraclostrobin, picoxystrobin, pyrametostrobin, pyraoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, fluxapyroxad, hexaconazole, ipconazole, metconazole, penconazole, propiconazole, proquinazid, prothioconazole, pyriofenone, tebuconazole, triticonazole, famoxadone, prochloraz, penthiopyrad and boscalid (nicobifen) (i.e. as Component (b) in compositions).

Preferred for better control of plant diseases caused by fungal plant pathogens (e.g., lower use rate or broader spectrum of plant pathogens controlled) or resistance management are mixtures of a compound of this invention with a fungicide selected from the group: azoxystrobin, kresoxim-methyl, trifloxystrobin, pyraclostrobin, picoxystrobin, pyrametostrobin, pyraoxystrobin, dimoxystrobin, metominostrobin/fenominostrobin, quinoxyfen, metrafenone, cyflufenamid, fenpropidine, fenpropimorph, cyproconazole, epoxiconazole, flusilazole, metconazole, propiconazole, proquinazid, prothioconazole, pyriofenone, tebuconazole, triticonazole, famoxadone and penthiopyrad.

As mentioned in the Summary of the Invention, one aspect of the present invention is a composition comprising (i.e. a mixture or combination of) a compound of Formula 1, an N-oxide, or a salt thereof, and at least one invertebrate pest control compound or agent (e.g., insecticide, acaricide). Of note is a composition comprising component (a) and at least one (i.e. one or more) invertebrate pest control compound or agent, which then can be subsequently combined with component (b) to provide a composition comprising components (a) and (b) and the one or more invertebrate pest control compounds or agents. Alternatively without first mixing with component (b), a biologically effective amount of the composition comprising component (a) with at least one invertebrate pest control agent can be applied to a plant or plant seed (directly or through the environment of the plant or plant seed) to protect the plant or plant seed from diseases caused by fungal pathogens and injury caused by invertebrate pests.

For embodiments where one or more of invertebrate pest control compounds are used, the weight ratio of these compounds (in total) to the component (a) compounds is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity.

Of note is a composition of the present invention which comprises in addition to a component (a) compound, alone or in combination with fungicidal component (b), at least one invertebrate pest control compound or agent selected from the group consisting of abamectin, acephate, acetamiprid, acetoprole, aldicarb, amidoflumet, amitraz, avermectin, azadirachtin, azinphos-methyl, bifenthrin, bifenazate, bistrifluoron, buprofezin, carbofuran, cartap, chinomethionat, chlorfenapyr, chlorfluazuron, chlorantraniliprole, chlorpyrifos, chlorpyrifos-methyl, chlorobenzilate, chromafenozide, clothianidin, cyantraniliprole, cyflumetofen, cyfluthrin, beta-cyfluthrin, cyhalothrin, gamma-cyhalothrin, lambda-cyhalothrin, cyhexatin, cypermethrin, cyromazine, deltamethrin, diafenthiuron, diazinon, dicofol, dieldrin, dienochlor, diflubenzuron, dimefluthrin, dimethoate, dinotefuran, diofenolan, emamectin, endosulfan, esfenvalerate, ethiprole, etoxazole, fenamiphos, fenazaquin, fenbutatin oxide, fenothiocarb, fenoxycarb, fenpropathrin, fenpyroximate, fenvalerate, fipronil, flonicamid, flubendiamide, flucythrinate, tau-fluvalinate, flufenerim, flufenoxuron, fonophos, halofenozide, hexaflumuron, hexythiazox, hydramethylnon, imicyafos, imidacloprid, indoxacarb, isofenphos, lufenuron, malathion, meperfluthrin, metaflumizone, metaldehyde, methamidophos, methidathion, methomyl, methoprene, methoxychlor, methoxyfenozide, metofluthrin, monocrotophos, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spiridiclofen, spiromesifen, spirotetramat, sulfoxaflor, sulprofos, tebufenozide, tebufenpyrad, teflubenzuron, tefluthrin, terbufos, tetrachlorvinphos, tetramethylfluthin, thiacloprid, thiamethoxam, thiodicarb, thiosultap-sodium, tolfenpyrad, tralomethrin, triazamate, trichlorfon, triflumuron, Bacillus thuringiensis subsp. aizawai, Bacillus thuringiensis subsp. kurstaki, nucleopolyhedro viruses, encapsulated delta-endotoxins of Bacillus thuringiensis, baculoviruses, entomopathogenic bacteria, entomopathogenic viruses and entomopathogenic fungi.

In certain instances, combinations of a compound of Formula 1 or an N-oxide or salt thereof (i.e. component (a)), alone or in mixture with fungicidal component (b), with other biologically active (particularly invertebrate pest control) compounds or agents (i.e. active ingredients) can result in a greater-than-additive (i.e. synergistic) effect. Reducing the quantity of active ingredients released in the environment while ensuring effective pest control is always desirable. When synergism of invertebrate pest control active ingredients occurs at application rates giving agronomically satisfactory levels of invertebrate pest control, such combinations can be advantageous for reducing crop production cost and decreasing environmental load.

One embodiment of invertebrate pest control agents (e.g., insecticides and acaricides) for mixing with compounds of Formula 1 (and N-oxides and salts thereof) include sodium channel modulators such as bifenthrin, cypermethrin, cyhalothrin, lambda-cyhalothrin, cyfluthrin, beta-cyfluthrin, deltamethrin, dimefluthrin, esfenvalerate, fenvalerate, indoxacarb, metofluthrin, profluthrin, pyrethrin and tralomethrin; cholinesterase inhibitors such as chlorpyrifos, methomyl, oxamyl, thiodicarb and triazamate; neonicotinoids such as acetamiprid, clothianidin, dinotefuran, imidacloprid, nitenpyram, nithiazine, thiacloprid and thiamethoxam; insecticidal macrocyclic lactones such as spinetoram, spinosad, abamectin, avermectin and emamectin; GABA (γ-aminobutyric acid)-regulated chloride channel blockers such as endosulfan, ethiprole and flpronil; chitin synthesis inhibitors such as buprofezin, cyromazine, flufenoxuron, hexaflumuron, lufenuron, novaluron, noviflumuron and triflumuron; juvenile hormone mimics such as diofenolan, fenoxycarb, methoprene and pyriproxyfen; octopamine receptor ligands such as amitraz; ecdysone agonists such as azadirachtin, methoxyfenozide and tebufenozide; ryanodine receptor ligands such as ryanodine, anthranilic diamides such as chlorantraniliprole, cyantraniliprole and flubendiamide; nereistoxin analogs such as cartap; mitochondrial electron transport inhibitors such as chlorfenapyr, hydramethylnon and pyridaben; lipid biosynthesis inhibitors such as spirodiclofen and spiromesifen; cyclodiene insecticides such as dieldrin; cyflumetofen; fenothiocarb; flonicamid; metaflumizone; pyrafluprole; pyridalyl; pyriprole; pymetrozine; spirotetramat; and thiosultap-sodium. One embodiment of biological agents for mixing with compounds of component (a) include nucleopolyhedro virus such as HzNPV and AfNPV; Bacillus thuringiensis and encapsulated delta-endotoxins of Bacillus thuringiensis such as Cellcap, MPV and MPVII; as well as naturally occurring and genetically modified viral insecticides including members of the family Baculoviridae as well as entomophagous fungi. Of note is a composition comprising component (a) and at least one additional biologically active compound or agent selected from the Invertebrate Pest Control Agents listed in Table D1 above.

The following Tests demonstrate the control efficacy of compounds of this invention on specific pathogens. The pathogen control protection afforded by the compounds is not limited, however, to these species. See Index Table A for compound descriptions. The following abbreviations are used in the Index Tables which follow: Me is methyl, Ph is phenyl, MeO is methoxy. The abbreviation “Ex.” stands for “Example” and is followed by a number indicating in which example the compound is prepared. “Cmpd No.” means compound number. Mass spectra (M.S.) are reported as the molecular weight of the highest isotopic abundance parent ion (M+1) formed by addition of H⁺ (molecular weight of 1) to the molecule, observed by mass spectrometry using atmospheric pressure chemical ionization (AP⁺).

INDEX TABLE A

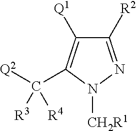

| Cmpd No. | R¹ | R² | R³ | R⁴ | Q¹ | Q² | M.S |
|---|---|---|---|---|---|---|---|
| 1 (Ex. 1) | H | Me | H | F | 2-Cl-4-F—Ph | 2,4-di-F—Ph | 367 |
| 2 (Ex. 2) | H | Me | H | Cl | 2-Cl-4-F—Ph | 2,4-di-F—Ph | 385 |
| 3 | H | Me | H | F | 2,6-di-F—Ph | 4-MeO-2,6-di-F—Ph | 383 |
| 4 | H | Me | H | F | 2,6-di-F—Ph | 2,4-di-F—Ph | 353 |
| 5 | H | Me | H | Cl | 2,6-di-F—Ph | 2,4-di-F—Ph | 369 |
| 6 | H | Me | H | F | 2-Cl-6-F—Ph | 2-Cl-4-F—Ph | 385 |
| 7 | H | Me | H | Cl | 2-Cl-6-F—Ph | 2-Cl-4-F—Ph | 401 |

### Biological Examples Of The Invention

General protocol for preparing test suspensions for Tests A-F: the test compounds were first dissolved in acetone in an amount equal to 3% of the final volume and then suspended at the desired concentration (in ppm) in acetone and purified water (50/50 mix) containing 250 ppm of the surfactant Trem® 014 (polyhydric alcohol esters). The resulting test suspensions were then used in Tests A-F. Each test was conducted in triplicate, and the results were averaged. Spraying a 200 ppm test suspension to the point of run-off on the test plants was the equivalent of a rate of 800 g/ha.

### Test A

The test suspension was sprayed to the point of run-off on tomato seedlings. The following day the seedlings were inoculated with a spore suspension of Botrytis cinerea (the causal agent of tomato botrytis) and incubated in saturated atmosphere at 20° C. for 48 h, and then moved to a growth chamber at 24° C. for 3 days, after which time visual disease ratings were made.

### Test B

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 5 days, after which time visual disease ratings were made.

### Test C

The test suspension was sprayed to the point of run-off on wheat seedlings. The following day the seedlings were inoculated with a spore suspension of Septoria nodorum (the causal agent of wheat glume blotch) and incubated in a saturated atmosphere at 24° C. for 48 h, and then moved to a growth chamber at 20° C. for 9 days, after which time visual disease ratings were made.

### Test D

The test suspension was sprayed to the point of run-off on wheat seedlings. The following day the seedlings were inoculated with a spore suspension of Septoria tritici (the causal agent of wheat leaf blotch) and incubated in saturated atmosphere at 24° C. for 48 h, and then the seedlings were moved to a growth chamber at 20° C. for 19 additional days, after which time visual disease ratings were made.

### Test E1

Wheat seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 2 days. At the end of this time the test suspension was sprayed to the point of run-off on the wheat seedlings, and then the seedlings were moved to a growth chamber at 20° C. for 4 days, after which time visual disease ratings were made.

### Test E2

The test suspension was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 6 days, after which time visual disease ratings were made.

### Test F

The test suspension was sprayed to the point of run-off on wheat seedlings. The following day the seedlings were inoculated with a spore dust of Blumeria graminis f. sp. tritici (also known as Erysiphe graminis f. sp. tritici, the causal agent of wheat powdery mildew) and incubated in a growth chamber at 20° C. for 8 days, after which time visual disease ratings were made.

Results for Tests A-F are given in Table A. In the Table, a rating of 100 indicates 100% disease control and a rating of 0 indicates no disease control (relative to the controls). A dash (-) indicates no test results.

TABLE A

| Cmpd | | | | | | | Test |
|---|---|---|---|---|---|---|---|
| No. | Test A | Test B | Test C | Test D | Test E1 | Test E2 | F |
| 1 | 100 | 83 | 0 | 98 | 40 | 99 | 69 |
| 2 | 99 | 0 | 0 | 100 | 0 | 94 | 0 |
| 3 | 100 | 46 | 0 | 100 | 98 | 100 | 99 |
| 4 | 99 | 98 | 0 | 52 | 66 | 18 | 0 |
| 5 | 100 | 100 | 89 | 100 | 9 | 100 | 100 |
| 6 | — | — | — | 96 | — | 98 | 87 |
| 7 | — | — | — | 100 | — | 100 | 100 |

“Cmpd No.” means compound number and refers to the same compound number as in Index Table A.

What is claimed is:

1. A compound selected from Formula 1, N-oxides and salts thereof, 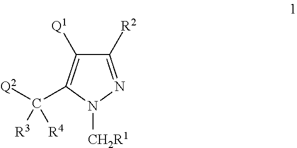
wherein
Q¹ is a phenyl ring or a naphthalenyl ring system, each ring or ring system optionally substituted with up to 5 substituents independently selected from R⁵; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2 S and up to 4 N atoms, wherein up to 3 carbon ring members are independently selected from C(═O) and C(═S), and the sulfur atom ring members are independently selected from S(═O)ᵤ(═NR⁶)ᵥ, each ring or ring system optionally substituted with up to 5 substituents independently selected from R⁵ on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
Q² is a phenyl ring or a naphthalenyl ring system, each ring or ring system optionally substituted with up to 5 substituents independently selected from R⁵; or a 5- to 6-membered saturated, partially unsaturated or fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2 S and up to 4 N atoms, wherein up to 3 carbon ring members are independently selected from C(═O) and C(═S), and the sulfur atom ring members are independently selected from S(═O)ᵤ(═NR⁶)ᵥ, each ring or ring system optionally substituted with up to 5 substituents independently selected from R⁵ on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C₁-C₁₂ alkyl, C₂-C₁₂ alkenyl, C₂-C₁₂ alkynyl, C₃-C₁₂ cycloalkyl or C₃-C₁₂ cycloalkenyl, each optionally substituted with up to 5 substituents independently selected from R⁵;
R¹ is H or CH₃;
R² is C₁-C₂ alkyl, halogen, cyano, cyanomethyl, halomethyl, hydroxymethyl, methoxy or methylthio; or cyclopropyl optionally substituted with up to 2 substituents independently selected from halogen and methyl;
R³ is H, halogen or C₁-C₄ alkyl;
R⁴ is halogen;
each R⁵ is independently selected from halogen, cyano, nitro, amino, methylamino, dimethylamino, formylamino, C₂-C₃ alkylcarbonylamino, C₁-C₄ alkyl, C₁-C₄ haloalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₂ alkylsulfonyloxy, C₁-C₂ haloalkylsulfonyloxy, C₃-C₄ cycloalkyl, C₃-C₇ cycloalkoxy, C₄-C₆ alkylcycloalkyl, C₄-C₆ cycloalkylalkyl, C₃-C₇ halocycloalkyl, C₂-C₄ alkenyl, C₂-C₄ alkynyl, hydroxy, formyl, C₂-C₃ alkylcarbonyl, C₂-C₃ alkylcarbonyloxy, —SF_{S}, —SCN, C(═S)NR⁷R⁸ or -U-V-T;
each R⁶ is independently H, cyano, C₁-C₃ alkyl or C₁-C₃ haloalkyl;
each R⁷ and R⁸ is independently H or CH₃;
each U is independently O, S(═O)_{w}, NR⁹ or a direct bond;
each V is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each T is independently cyano, NR^{10a}R^{10b}, OR¹¹ or S(═O)_{y}R¹²
each R⁹ is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{10a} and R^{10b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{10a} and R^{10b} attached to the same nitrogen atom are taken together with the nitrogen atom to form a 3- to 6-membered heterocyclic ring, the ring optionally substituted with up to 5 substituents independently selected from R¹³;
each R¹¹ and R¹² is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R¹³ is independently halogen, C₁-C₆ alkyl, C₁-C₆ haloalkyl or C₁-C₆ alkoxy;
each u and v are independently 0, 1 or 2 in each instance of S(═O)ᵤ(═NR⁶)ᵥ, provided that the sum of u and v is 0, 1 or 2;
each w is independently 0, 1 or 2; and
each y is independently 0, 1 or 2.

2. A compound of claim 1 wherein
Q¹ is phenyl, pyridinyl, pyrimidinyl, pyrazinyl or pyridazinyl, each substituted with from 1 to 4 substituents independently selected from R⁵; provided that when an R⁵ substituent is located at a meta position, then said R⁵ substituent is selected from F, Cl, Br and cyano;
Q² is phenyl, pyridinyl, pyrimidinyl, pyrazinyl or pyridazinyl, each substituted with 1, 2 or 3 substituents independently selected from R⁵, provided that when an R⁵ substituent is located at a meta position, then said R⁵ substituent is selected from F, Cl, Br and cyano;
R² is C₁-C₂ alkyl, Cl or Br; and
each R⁵ is independently selected from halogen, cyano, nitro, amino, methylamino, dimethylamino, C₁-C₄ alkyl, C₁-C₄ haloalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₃-C₄ cycloalkyl, C(═S)NH₂ and -U-V-T.

3. A compound of claim 2 wherein:
Q¹ is phenyl or pyridinyl, each substituted with 1, 2 or 3 substituents independently selected from R⁵;
Q² is phenyl or pyridinyl, each substituted with 1, 2 or 3 substituents independently selected from R⁵;
R² is CH₃, Cl or Br;
R³ is H, F, Cl, Br or CH₃;
R⁴ is F, Cl or Br;
each R⁵ is independently selected from halogen, cyano, nitro, C₁-C₄ alkyl, C₁-C₄ haloalkyl, C₁-C₃ alkoxy, C₁-C₃ haloalkoxy and -U-V-T;
each U is independently O or NH;
each V is C₂-C₄ alkylene;
each T is independently NR^{10a}R^{10b} or OR¹¹;
each R^{10a} and R^{10b} is independently H, C₁-C₆ alkyl or C₁-C₆ haloalkyl; and
each R¹¹ is independently H, C₁-C₆ alkyl or C₁-C₆ haloalkyl.

4. A compound of claim 3 wherein
at least one of Q¹ and Q² is phenyl substituted with 2 or 3 substituents independently selected from R⁵;
R¹ is H;
R² is CH₃;
R³ is H, F, Cl or Br; and
each R⁵ is independently selected from halogen, cyano, C₁-C₃ alkyl, C₁-C₃ haloalkyl, C₁-C₃ alkoxy and C₁-C₃ haloalkoxy.

5. A compound of claim 4 wherein
Q¹ is phenyl substituted at the 2-, 4- and 6-positions with substituents independently selected from R⁵; or phenyl substituted at the 2- and 4-positions with substituents independently selected from R⁵; or phenyl substituted at the 2- and 6-positions with substituents independently selected from R⁵;
Q² is phenyl substituted at the 2-, 4- and 6-positions with substituents independently selected from R⁵; or phenyl substituted at the 2- and 4-positions with substituents independently selected from R⁵; or phenyl substituted at the 2- and 6-positions with substituents independently selected from R⁵;
R³ is H, F or Cl;
R⁴ is F or Cl; and
each R⁵ is independently selected from F, Cl, Br, cyano, C₁-C₂ alkyl, C₁-C₂ haloalkyl, C₁-C₂ alkoxy and C₁-C₂ haloalkoxy.

6. A compound of claim 5 wherein
R³ is H; and
each R⁵ is independently selected from F, Cl, Br, cyano, methyl, C₁-C₂ alkoxy and fluoromethoxy.

7. A compound of claim 6 wherein
each R⁵ is independently selected from F, Cl, Br, cyano and methoxy.

8. A compound of claim 1 which is selected from the group:
4-(2-chloro-4-fluorophenyl)-5-[(2,4-difluorophenyl)fluoromethyl]-1,3-dimethyl-1H-pyrazole,
5-[chloro(2,4-difluorophenyl)methyl]-4-(2-chloro-4-fluorophenyl)-1,3-dimethyl-1H-pyrazole,
5-[bromo(2,4-difluorophenyl)methyl]-4-(2-chloro-4-fluorophenyl)-1,3-dimethyl-1H-pyrazole,
4-(2-chloro-4-fluorophenyl)-5-[(2,4-difluorophenyl)difluoromethyl]-1,3-dimethyl-1H-pyrazole,
4-(2-chloro-4-fluorophenyl)-5-[dichloro (2,4-difluorophenyl)methyl]-1,3-dimethyl-1H-pyrazole,
4-(2-chloro-4-fluorophenyl)-5-[dibromo (2,4-difluorophenyl)methyl]-1,3-dimethyl-1H-pyrazole,
5-[chloro(2,4-difluorophenyl)fluoromethyl]-4-(2-chloro-4-fluorophenyl)-1,3-dimethyl-1H-pyrazole,
5-[1-chloro-1-(2,4-difluorophenyl)ethyl]-4-(2-chloro-4-fluorophenyl)-1,3-dimethyl-1H-pyrazole,
4-(2-chloro-4-fluorophenyl)-5-[1-(2,4-difluorophenyl)-1-fluoroethyl]-1,3-dimethyl-1H-pyrazole, and
5-[1-bromo-1-(2,4-difluorophenyl)ethyl]-4-(2-chloro-4-fluorophenyl)-1,3-dimethyl-1H-pyrazole.

9. A fungicidal composition comprising (a) a compound of claim 1; and (b) at least one other fungicide.

10. A fungicidal composition comprising: (a) a compound of claim 1; and (b) at least one additional component selected from the group consisting of surfactants, solid diluents and liquid diluents.

11. A method for controlling plant diseases caused by fungal plant pathogens comprising applying to the plant or portion thereof, or to the plant seed, a fungicidally effective amount of a compound of claim 1.

12. A composition comprising a compound of claim 1, and at least one invertebrate pest control compound or agent.
